(19) United States
(12) Patent Application Publication
(10) Pub. No.: US 2026/0096956 A1
(43) Pub. Date: Apr. 9, 2026

(54) NON-WHITENING MINERAL SUNSCREEN

(71) Applicant: L'OREAL, Paris (FR)

(72) Inventors: Anil SHAH, East Windsor, NJ (US); Brian BODNAR, Manasquan, NJ (US)

(73) Assignee: L'OREAL, Paris (FR)

(21) Appl. No.: 18/906,221

(22) Filed: Oct. 4, 2024

(51) Int. Cl. A61K 8/06; A61Q 17/04

(52) U.S. Cl. CPC A61K 8/062; A61Q 17/04; A61K 2800/30; A61K 2800/413; A61K 2800/48; A61K 2800/522

(57) ABSTRACT

A mineral sunscreen composition is provided as a stable oil-in-water emulsion that includes aqueous and oil phases, including a UV absorbing system that includes at least one physical UV attenuating material, at least one aqueous phase hydrophobically-modified polymeric thickener having carboxylic acid groups, and an emulsification system that includes at least one emulsifier having a high HLB, and at least one emulsifier having a low HLB. The composition includes a solvent system that includes water phase solvents and oil phase solvents comprising one or a combination of oil/fatty compounds and silicones.

### FIELD OF THE INVENTION

The present disclosure is directed to skin care compositions that provide UV protection, in particular that does not lighten or whiten on application.

### BACKGROUND OF THE INVENTION

The photoprotection of keratinous substrates, including both skin and hair, is considered by many to be necessary in order to facilitate protection from sun-damage, sunburn, photo-aging, as well as to decrease the chances of skin cancer development caused by exposure to ultraviolet (“UV”) radiation.

The negative effects of exposure to ultraviolet (“UV”) light are well known. By way of example only, exposure to sunlight can cause damage such as sunburn to the skin and dry out hair, making it brittle. In particular, when skin is exposed to UV light (wavelengths from about 290 nm to about 400 nm), long term damage can lead to serious conditions such as skin cancer. Further, UV light contributes to aging by causing free radicals to form in the skin. Free radicals include, for example, singlet oxygen, hydroxyl radical, the superoxide anion, nitric oxide, and hydrogen radicals. Free radicals attack DNA, membrane lipids and proteins, generating carbon radicals. These in turn react with oxygen to produce a peroxyl radical that can attack adjacent fatty acids to generate new carbon radicals. This cascade leads to a chain reaction producing lipid peroxidation products. This process is commonly referred to as photo-aging. Damage to the cell membrane results in loss of cell permeability, increased intercellular ionic concentration, and decreased ability to excrete or detoxify waste products. The end result is loss of skin elasticity, including effects on the biomechanical properties, appearance, and health of the epidermis, erythema (sunburn), immediate skin darkening, appearance of wrinkles leading to the premature aging of the skin (i.e. photoaging), and a variety of other skin health-related concerns which may include melanoma or other skin cancers associated with UV/sun exposure.

Sunscreens can be used to protect against UV damage and delay the signs of photo-aging. One current trend in photoprotection are mineral sunscreens which provide desirable efficacy and good sensory properties such as a fluid texture and non-greasy feel when applied. Consumers prefer mineral sunscreen compositions to include natural and not irritating components that appear natural (unnoticeable) once applied. The degree of UV protection afforded by a mineral sunscreen composition is directly related to the amount and type of UV filters contained therein. Of course, relatively high concentrations of mineral/inorganic UV filters (also referred to as physical UV attenuating materials) are required to achieve higher efficacy. It is known in the art to that there are challenges to providing mineral-based sunscreen products having a high Sun Protection Factor (SPF). Indeed, higher concentrations of physical UV attenuating materials typically results in whitening upon application to skin which is problematic for consumers. Further, it is difficult to develop formulas with higher concentrations of physical UV attenuating materials that are shelf stable, are capable of maintaining SPF benefit, and are not whitening on darker skin.

What is needed is a composition that provides the level of protection achievable with physical UV attenuating materials in a formulation that provides a pleasant texture, non-whitening with the same or higher levels of SPF as compared with sunscreens having high amounts of physical UV attenuating materials that can be unstable or whiten skin.

### BRIEF SUMMARY OF THE INVENTION

The instant disclosure provides a mineral sunscreen composition that includes physical UV attenuating materials, which are known to be non-irritating, natural, and gentle to the skin that confers stable sun protection and emulsion structural stability in the presence of high amounts of physical UV attenuating materials selected from metal oxide UV filters. In some embodiments, provided is a mineral sunscreen composition that is a stable oil-in-water emulsion (as further defined herein) that includes aqueous and oil phases.

The mineral sunscreen composition overcomes challenges known in the art wherein high levels of physical UV attenuating materials have been shown to be difficult to stabilize in formulations, and wherein in high amounts the physical UV attenuating materials lighten or whiten the skin on application.

In an embodiment, the disclosure provides a mineral sunscreen composition in the form of a broad spectrum, low viscosity, oil-in-water emulsion comprising aqueous and oil phases, the mineral sunscreen composition comprising: a UV absorbing system comprising at least one physical UV attenuating material, the at least one physical UV attenuating material comprising zinc oxide particles having a mean size in a range from about 15 nm to about 35 nm, the zinc oxide particles coated with triethoxycaprylylsilane; an emulsification system comprising at least one high HLB emulsifier having an HLB of greater than 13, and at least one low HLB emulsifier having an HLB of less than 6; at least one aqueous phase hydrophobically-modified anionic polymeric thickener comprising carboxylic acid residues; wherein the combined high HLB and low HLB emulsifiers to hydrophobically-modified anionic polymeric thickener are present in a ratio in a range from about 1.0:0.05 to about 1.0:0.15, and wherein the mineral sunscreen composition demonstrates an SPF that is at least 40, a UVA1:UV ratio that is at least 0.7, and a critical wavelength that is greater than 370 nm.

In some embodiments, the mineral sunscreen composition has one or more of the following properties:
(a) a critical wavelength as determined by FDA's critical wavelength procedures of at least 370 nm;
(b) an SPF value of at least 15, and, in some embodiments, at least 30, and, in some embodiments, at least 40 and, in some embodiments, at least 50;
(c) a UVA1/UV ratio of 0.7 or higher.

In some embodiments, the mineral sunscreen composition has one or more of the following properties:
(a) wherein the mineral sunscreen composition excludes organic UV filters.
(b) wherein the zinc oxide particles are selected from the group consisting of zinc oxide (and) triethoxycaprylylsilane, having a particle size of about 20 nm, zinc oxide (and) silica (and) hydrogen dimethicone, having a particle size of about 25 nm, zinc oxide (and) caprylic/capric triglyceride (and) polyhydroxystearic acid (and) isostearic acid, having a particle size of about 60 nm, zinc oxide (and) C12-15 alkyl benzoate (and) triethoxycaprylylsilane (and) polyhydroxystearic acid, having a particle size of about 30 nm, and combinations thereof.
(c) wherein the at least one physical UV attenuating material is present in the mineral sunscreen composition in the range from about 10% to about 25%, all amounts based on the total weight of the mineral sunscreen composition.
(d) the aqueous phase hydrophobically-modified anionic polymeric thickener comprises acrylates/C10-30 alkyl acrylate crosspolymer.
(e) the emulsification system comprises glyceryl stearate, steareth-2, steareth-20, and polysorbate 20.
(f) comprising a rheology modifier selected from the group consisting of xanthan gum, cellulose gum, biosaccharide gum, carrageenan, gellan gum, or associative thickeners such as like Sepinov EMT10™ or Sepigel™, Aristoflex™, and combinations thereof.
(g) comprising at least one antioxidant comprising one or more of diethylhexyl syringylidenemalonate and ethylhexyl methoxycrylene.
(h) comprising an oil phase solvent system comprising one or a combination of oil/fatty compounds and silicones selected from caprylic/capric triglyceride, cetearyl isononanoate, cetearyl alcohol, dicaprylyl carbonate, isohexadecane, C15-19 alkane, dimethicone, and combinations thereof.
(i) comprising one or more additives selected from the group consisting of skin conditioners, hydrating agents, skin actives, preservatives, antimicrobials, powders, fillers, and combinations thereof.
(j) comprising one or more additives selected from the group consisting of triethylhexanoin, glycerin, propanediol, tocopherol, niacinamide, hydroxyacetophenone, citric acid, chlorphenesin, caprylyl glycol, aluminum stearate, alumina, silica, cellulose, and combinations thereof.
(k) wherein the components of the aqueous phase and components of the oil phase, excluding the physical UV attenuating materials and any powders, respectively, are present in the mineral sunscreen composition at a weight ratio of aqueous components to oil components in a range from about 1:1 to about 4:1.

The present invention is also directed to a method for forming a metastable structured emulsion formed through the interaction of the carboxylic acid residues of an aqueous phase hydrophobically-modified anionic polymeric thickener comprising acrylates/C10-30 alkyl acrylate crosspolymer with a physical UV attenuating material comprising one or more of zinc oxide and/or titanium dioxide, more particularly a coated form of one or more of zinc oxide and/or titanium dioxide wherein the coating is selected from hydrated silica, triethoxysilylethyl polydimethylsiloxyethyl, hexyl dimethicone and/or hydrogen dimethicone, triethoxycaprylysilane, and combinations thereof, which emulsion provides a high SPF that does not whiten on application.

In an embodiment, the disclosure provides a metastable structured emulsion comprising an aqueous phase hydrophobically-modified anionic polymeric thickener comprising acrylates/C10-30 alkyl acrylate crosspolymer and a physical UV attenuating material comprising one or more of zinc oxide and/or titanium dioxide, the one or more of zinc oxide and/or titanium dioxide comprising a coating selected from hydrated silica, triethoxysilylethyl polydimethylsiloxyethyl, hexyl dimethicone, hydrogen dimethicone, triethoxycaprylysilane, or a combination thereof, the metastable structured emulsion formed through the interaction of carboxylic acid residues of the aqueous phase hydrophobically-modified polymeric thickener with the coated zinc oxide and/or titanium dioxide, which emulsion provides a high SPF that does not whiten on application by rubbing on a substrate such as skin.

In an embodiment, the disclosure provides a mineral sunscreen composition in the form of a broad spectrum, low viscosity, oil-in-water emulsion comprising aqueous and oil phases, the mineral sunscreen composition comprising: a UV absorbing system comprising at least one physical UV attenuating material, the at least one physical UV attenuating material comprising zinc oxide particles having a primary particle size in a range from about 15 nm to about 35 nm, the zinc oxide particles coated with triethoxycaprylylsilane, and titanium dioxide; an emulsification system comprising at least one high HLB emulsifier having an HLB of greater than 13 comprising steareth-20 and polysorbate 20 and at least one low HLB emulsifier having an HLB of less than 6, comprising glyceryl stearate and steareth-2; at least one polymeric dispersant comprising polyhydroxystearic acid; at least one aqueous phase hydrophobically-modified anionic polymeric thickener comprising acrylates/C10-30 alkyl acrylate crosspolymer; at least one rheology modifier comprising xanthan gum; at least one antioxidant comprising diethylhexyl syringylidenemalonate and ethylhexyl methoxycrylene; an oil phase solvent system comprising caprylic/capric triglyceride, cetearyl isononanoate, cetearyl alcohol, dicaprylyl carbonate, isohexadecane, C15-19 alkane, and dimethicone; and one or more additives selected from the group consisting of triethylhexanoin, glycerin, propanediol, tocopherol, niacinamide, hydroxyacetophenone, citric acid, chlorphenesin, caprylyl glycol, aluminum stearate, alumina, silica, cellulose, and combinations thereof, wherein the combined high HLB and low HLB emulsifiers to hydrophobically-modified anionic polymeric thickener are present in a ratio in a range from about 1.0:0.05 to about 1.0:0.15, and wherein the mineral sunscreen composition demonstrates an SPF that is at least 40, a UVA-I/UV ratio that is at least 0.7, and a critical wavelength that is greater than 370 nm.

The present invention is also directed to a method of inhibiting UV radiation from contacting a keratinous substrate by applying the above-disclosed mineral sunscreen composition onto a surface of the keratinous substrate.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

Provided herein is a light, mineral sunscreen composition that is pleasant to the touch with good spreadability and that does not lighten or whiten the skin. The mineral sunscreen composition includes separate oil and water phases that provide an oil-in-water emulsion.

The inventors have overcome challenges encountered in the art and provide a fluid stable emulsion sunscreen formulation which provides SPF (in-vivo) of at least 40 and, in some embodiments, up to at least 50 with no whitening on skin, including dark skin, and which provides good spreadability and textural aesthetics. The inventive composition is formulated as a mineral-based sunscreen in which solid particles of mineral-based UV filters include, for example, zinc oxide and titanium dioxide in the presence of an aqueous phase hydrophobically-modified anionic polymeric thickener comprising acrylates/C10-30 alkyl acrylate crosspolymer and an emulsifier system.

Without being bound by theory, the present invention describes an oil-in-water emulsion, stabilized by the emulsifier system and the combination of physical UV attenuating materials and the aqueous phase hydrophobically-modified anionic polymeric thickener. In the present invention, it is believed that the carboxylic acid residues within the aqueous phase hydrophobically-modified anionic polymeric thickener interact with the physical UV attenuating material to form an additional metastable structuring of the emulsion which is easily broken upon application to a keratinous substrate, and which may aid the spreadability, homogeneity, UV filtering effect, and non-whitening appearance on skin of the mineral sunscreen composition.

As exemplified herein, the inventors have shown a unique and unexpected effect achieved using physical UV attenuating material in the form of one or more metal oxide UV filters, in particular zinc oxide and titanium dioxide which associate with carboxylic acid moieties of an aqueous phase hydrophobically-modified anionic polymeric thickener to provide a structurally stable emulsion which confers high SPF even as compared with compositions including greater amounts of the metal oxide UV filters, and which do not whiten on application. The results show a surprising interaction between the one or more metal oxide UV filters and the aqueous phase hydrophobically-modified anionic polymeric thickener which is evidenced by rheology data. The instant exemplified formulations also show that an emulsifier system comprising at least one high HLB emulsifier having an HLB of greater than 13 and at least one low HLB emulsifier having an HLB of less than 6, combined with the one or more metal oxide UV filters and the aqueous phase hydrophobically-modified anionic polymeric thickener provide a shelf stable emulsion.

In various embodiments, the mineral sunscreen compositions are provided as an oil-in-water emulsion, wherein the discrete phases that include the aqueous/water soluble components of the water phase and the non-aqueous/water insoluble components of the oil phase, excluding the physical UV attenuating materials and any powders, are each present in the mineral sunscreen composition at a weight ratio of aqueous to non-aqueous in a range from about 1:1 to about 4:1.

### Definitions

A UV absorbing system essentially containing at least one “physical UV attenuating material” as that term is used herein means that compositions of the disclosure contain less than 3% UV filters other than physical UV attenuating material(s), in particular less than 3% of UV organic filters, meaning that within this definition as subcategories are compositions containing less than 2% UV filters other than physical UV attenuating material(s), in particular less than 2% of UV organic filters, and less than 1% UV filters other than physical UV attenuating material(s), in particular less than 1% of UV organic filters, as well as being “free of UV filters other than physical UV attenuating material,” “substantially free of UV filters other than physical UV attenuating material,” and “devoid of UV filters other than physical UV attenuating material” as defined herein.

“Antioxidant” or “SPF boosting antioxidant” refers to a material which demonstrates antioxidant properties and as used according to the disclosure increases the UV absorption of the mineral UV sunscreen material when the two are intermixed in the mineral sunscreen composition by refracting UV radiation, thereby increasing the effective path length of the UV radiation through the mineral sunscreen composition. An antioxidant, which is not a UV filter as defined herein, is thus a compound present in an amount which increases the UV efficacy of a composition containing UV filters compared to the UV efficacy of the same composition containing UV filters but not containing the antioxidant. The antioxidant is effective in increasing the UV efficacy as measured by at least one of SPF, UVAPF, Critical Wavelength, UVA-I/UV ratio, and/or UVAPF/SPF ratio.

“UVAPF” (UVA protection factor) relates to an index characterizing the protection against UVA provided by a composition. For example, the UVAPF index can be measured in vivo according to the “PPD” (Persistent Pigment Darkening) method in the ISO-24442:2022 protocol, measuring observed skin color 2 to 4 hours after UVA exposure. Also, it can be determined according to FDA protocols, again as described in 21 C.F.R. Part 352 Subpart D § 352.72 as discussed above in connection with SPF.

“Sun Protection Factor” or “SPF” (Sun Protection Factor) measures the level of protection against erythema provided by a composition. “SPF” is a known term in the sunscreen art and is defined, for example, in A new substrate to measure sunscreen protection factors throughout the ultraviolet spectrum, J. Soc. Cosmet. Chem., 40, 127-133 (May/June 1989). “SPF” means and refers to a value expressed mathematically by the ratio of the irradiation time necessary to attain the erythemogenic threshold with the UV screening agent to the time necessary to attain the erythemogenic threshold without UV screening agent. SPF generally provides information about the skin's resistance to ultraviolet B (UVB) radiation from the sun. Thus, the SPF value corresponds to the ratio between the minimum erythemal dosage (MED) measured wearing the composition and the MED measured with bare skin. The SPF rating system has been developed to provide consumer guidance in selecting sunscreens. All SPF and UV-A ratings are provided on the basis of in-vivo value unless otherwise indicated.

The evaluation of the SPF (Sun Protection Factor) can be performed, for example, in vitro with spectrophotometer by Labsphere (North Sutton, NH, USA). In such an evaluation, the plate is the material on which the tested composition is applied. For such an evaluation, polymethylmethacrylate (PMMA) plates can be used. An example of an acceptable protocol is in the process of ISO accreditation under the name ISO Committee Draft 23675.

The evaluation of the SPF can also be performed in-vivo according to the ISO 24444:2019 protocol “Cosmetics-Sun protection test methods-In-vivo determination of the SPF.” Also, it can be determined according to FDA protocols, as described in the document “Labeling and Effectiveness Testing: Sunscreen Drug Products for Over-the-Counter Human Use” published in the US Federal Register on Jul. 5, 2011 (https://www.federalregister.gov/d/2011-14766); 21 C.F.R. Part 352 Subpart D § 352.72, updated and revised by the 2011 publication in the Federal Register.

All SPF and UV-A ratings are provided on the basis of in-vitro value unless otherwise indicated.

“Critical wavelength” is an absorption spectrum of a sunscreen composition characterized by an index, namely a wavelength, where the integral of the spectral absorbance curve reached 90% of the integral from 290 nm to 400 nm. The critical wavelength is used to determine the breadth of UV protection.

“Hydrophilic-Lipophilic Balance” or “HLB,” refers to an empirical expression for the relationship of the hydrophilic and hydrophobic groups of an emulsifier. This term is well known to those skilled in the art. See, e.g., “The HLB system. A time-saving guide to Emulsifier Selection” (Pub: ICI Americas Inc., 1984) and US2006/0217283 at [0053]. “High HLB” for an emulsifying emulsifier in an O/W emulsion of the invention means an HLB of equal to greater than about 9. “Low HLB” for an emulsifying emulsifier for an oil-in-water emulsions (“O/Ws”) emulsion of the invention means an HLB that is equal to or less than about 6. The HLB system is particularly useful to prepare O/W and water-in-oil emulsions (“W/Os”) wherein it is customarily understood that W/Os often employ low HLB emulsifiers, having an HLB value of equal to or less than 6 and O/Ws often employ high HLB emulsifiers having an HLB value equal to or greater than 9. The total HLB of emulsifiers can be calculated by taking the weight percent of each emulsifier of the emulsifier mixture and multiplying the weight percent by the HLB to obtain a contribution of each individual emulsifier on the HLB. The individual contributions are then added together to obtain the total HLB of the emulsifier mixture. HLBs for individual emulsifiers can be found in well-known literature in the art or may be provided by an emulsifier supplier.

“Stable” means the emulsion can be formed (i.e., does not fail to form upon combination of the ingredients), and remains fluid/liquidous without phase separation that cannot be reestablished by agitation, such stability being demonstrated over the stability monitoring period without appreciable color and/or odor change, and the water-soluble active ingredients remain solubilized in the water phase without crystallization or precipitation out of the emulsion, and the mineral oxide material(s) remain in association within the emulsion. In the present application, the term “ambient temperature” means a temperature of about 25° C.

“Passivated” as used in connection with the description of physical UV attenuating material herein refers to a material which has been treated in a way such that the potential to release ionic species when in contact with water is reduced compared to the same non-passivated material.

“Primary Particle” as used in connection with the description of physical UV attenuating material herein means inorganic or organic particles (structures) which can be held together via molecular or atomic bonding to form a physical UV attenuating material.

“Primary particle size” means the size of an unaggregated primary particle in a physical UV attenuating material.

### UV Absorbing System

The mineral sunscreen composition according to the disclosure includes at least one inorganic or mineral based UV filters in an UV absorbing system. According to the present disclosure, the “Physical ultraviolet (UV)-attenuating material” refers to solid inorganic ingredients which absorb incoming ultraviolet (UV)-light, and may optionally scatter incoming ultraviolet (UV)-light, when present in compositions disclosed herein. The physical UV-attenuating material and, in some embodiments, comprises metal oxide(s) such as, for example, oxides of titanium, chromium, zinc, tin, alumina, cerium, and/or iron. Specific examples of suitable metal oxide(s) include, but are not limited to, at least one metal oxide selected from the group consisting of titanium dioxide, zinc oxide, iron oxide, chromium oxide, tin oxide, alumina, cerium oxide, and mixtures thereof.

The physical UV-attenuating material may be subjected to a surface treatment agent to improve sensory, performance, and/or compatibility of the compositions disclosed herein. Suitable surface treatment agents may include hydrophobic or hydrophilic surface treatment agents such as, for example, those described in Cosmetics & Toiletries, February 1990, Vol. 105, pp. 53-64, including but not limited to specific examples such as amino acids, beeswax, fatty acids, fatty acid salts, fatty alcohols, anionic surfactants, lecithin, lecithin derivatives, metal alkoxides, polyethylene, silicones, proteins, alkanolamines, silicon oxides, metal oxides, sodium hexametaphosphate, alumina, and/or glycerol.

In some embodiments, as exemplified herein, and in some preferred embodiments, the physical UV-attenuating material comprises one or more of zinc oxide and/or titanium dioxide.

In some embodiments, the physical UV-attenuating material is not passivated.

In some embodiments, the physical UV-attenuating material may be passivated.

In some embodiments, the physical UV-attenuating material comprising one or more of zinc oxide or titanium dioxide may be passivated.

As described herein, the UV absorbing system may be devoid of organic UV filters, and may in some embodiments, include organic UV filters in an amount that is not greater than 3% by weight of the composition.

In some embodiments, the mineral sunscreen composition may include organic UV filters and may further include one or more SPF boosting antioxidants.

Examples of suitable UV filters include, but are not limited to, UV filters that are active in UV-A and/or UV-B regions, that may be water-soluble, fat-soluble or insoluble in commonly used cosmetic solvents, and may be inorganic or organic. UV-A filters comprise groups of compounds which absorb light predominantly in the range of wavelengths 400 nm to 320 nm (UV-A) and UV-B filters comprise groups of compounds which absorb light predominantly in the range of wavelengths 400 nm to 320 nm 320 nm to 280 nm (UV-B). According to an embodiment of the disclosure, UV-A and UV-B can be two separate UV filters or they can be one UV filter with both UV-A and UV-B sun protection factor.

In some embodiments, the UV absorbing system includes a combination of physical UV attenuating materials.

In some embodiments, the UV absorbing system includes zinc oxide.

In some embodiments, the mineral sunscreen compositions include a mixture of at least two or more of physical UV attenuating materials selected from zinc oxide and titanium dioxide.

In some embodiments as exemplified herein, the mineral sunscreen composition includes zinc oxide (˜10-17.5%), and titanium dioxide (˜1.5-6.5%), all amounts by weight, based on the weight of the composition.

In some embodiments as exemplified herein, the mineral sunscreen composition includes zinc oxide (˜15%), and titanium dioxide (˜2.7%), all amounts by weight, based on the weight of the composition.

In some embodiments as exemplified herein, the mineral sunscreen composition includes zinc oxide (˜12.4%), and titanium dioxide (˜4.1%), all amounts by weight, based on the weight of the composition.

In some embodiments as exemplified herein, the mineral sunscreen composition includes zinc oxide (˜10.2%), and titanium dioxide (˜1.8%), all amounts by weight, based on the weight of the composition.

In some embodiments as exemplified herein, the mineral sunscreen composition includes zinc oxide (˜13%), and titanium dioxide 0%), all amounts by weight, based on the weight of the composition.

In some embodiments as exemplified herein, one or both of the zinc oxide and titanium dioxide comprise a coating which comprises one or more of triethoxycaprylylsilane and triethylhexanoin.

In some embodiments as exemplified herein, the coatings which comprise triethoxycaprylylsilane (˜0.5-3%), all amounts by weight, based on the weight of the composition.

In some embodiments as exemplified herein, the coatings which comprise triethoxycaprylylsilane (˜0.5-3%), and triethylhexanoin (˜0.5-3.0%), all amounts by weight, based on the weight of the composition.

In some particular embodiments, the amount of an physical UV attenuating material present in the mineral sunscreen composition is at least about 5% by weight, and, in some embodiments, at least about 10% by weight, and, in some embodiments, at least about 12% by weight, and, in some embodiments, at least about 14% by weight, and, in some embodiments, at least about 15% by weight, with the upper end of the range of the at least one physical UV attenuating material present and, in some embodiments, being about 40% by weight (e.g., about 5% to about 40%, about 10% to about 40%, about 12% to about 40%, etc.), and, in some embodiments, about 30% by weight (e.g., about 5-% to about %, about 10-% to about %, about 12% to about 30%, etc.), and, in some embodiments, about 25% by weight (e.g., about 5% to about 25%, about 10% to about 25%, about 12% to about 25%, etc.), and, in some embodiments, about 20% by weight (e.g., about 5% to about 20%, about 10% to about 20%, about 12% to about 20%, etc.), with all weights being based on the total weight of the composition.

According to preferred embodiments, embodiments of the mineral sunscreen composition contain a UV absorbing system essentially containing physical UV attenuating material(s) as defined herein.

Thus, any one or combination of physical UV attenuating material may be present, by weight, based on the total weight of the mineral sunscreen composition, from about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40 percent, including increments and ranges therein and there between, based upon the total weight of the mineral sunscreen composition.

### Embodiments of Physical Ultraviolet Attenuating Materials

According to a first embodiment, the physical UV-attenuating material comprises at least zinc oxide. Zinc oxide may be present in any form (e.g., wurtzite or zincblende form) in the mineral sunscreen composition. Further, the zinc oxide can be treated (coated) or untreated.

According to a second embodiment, the physical UV-attenuating material comprises at least titanium dioxide. Titanium dioxide may be present in any form. Further, the TiO₂ can be treated (coated) or untreated.

According to a third embodiment, the physical UV-attenuating material comprises at least zinc oxide and titanium dioxide, wherein either or both may be present in any form and may be treated (coated) or untreated. In some particular embodiment, only one of the physical UV-attenuating materials is a coated oxide.

According to a first embodiment, the physical UV-attenuating material comprises titanium dioxide. Titanium dioxide may be present in any form. Further, the TiO₂ can be treated (coated) or untreated.

In some embodiments, the physical UV-attenuating material comprises one or more of zinc oxide.

In some embodiments, the zinc oxide may be passivated.

In some embodiments, the physical UV-attenuating material comprises one or

more of titanium dioxide, and, in some embodiments, the zinc oxide may be passivated.

In some embodiments, the mean primary particle size of the physical UV attenuating material is from 1 nm to 500 nm, and, in some embodiments, from 5 nm to 250 nm, and, in some embodiments, from 10 nm to 100 nm, and, in some embodiments, from 20 nm to 50 nm, including all ranges and subranges therebetween such as, for example, 25 nm to 40 nm, 10 nm to 75 nm, and 15 nm to 150 nm.

In some embodiments, the coated pigments are more particularly titanium oxides that have been coated:
with hydrated silica, such as the product MT-100WP from Tayca,
with silica and iron oxide, such as the product Sunveil FR from Ikeda,
with silica and alumina, such as the products MT-500SAR and MT-100SAR from Tayca and Tioveil™ AQ-N from Croda,
with alumina, such as the product TTO-55 (A) R from Ishihara,
with alumina and aluminium stearate, such as the products MT-100TV®, MT-100ZR and MT-01® from Tayca, the product Solaveil™ CT100R from Croda and the product Eusolex T-AVO® from Merck,
with silica, alumina and alginic acid, such as the product MT-100AQR from Tayca,
with alumina and aluminium laurate,
with iron oxide and iron stearate,
with zinc oxide and zinc stearate,
with silica and alumina and treated with a silicone, such as the products MTY-500SAS® or Microtitanium Dioxide MT-100SAS® from Tayca,
with silica, alumina, and aluminium stearate and treated with a silicone,
with silica and treated with a silicone,
with silica and treated with a silicone, such as the product TTO-55 (S)® from Ishihara,
with triethanolamine,
with stearic acid, such as the product TTO-55 (C)® from Ishihara,
with sodium hexametaphosphate,
with TiO2 treated with octyltrimethylsilane,
with TiO2 treated with a polydimethylsiloxane,
with anatase/rutile TiO2 treated with a polydimethylhydrogenosiloxane,
TiO2 coated with triethylhexanoin, with aluminium stearate and with alumina sold under the trade name Solaveil™ CT-200 by Croda,
TiO2 coated with aluminium stearate, with alumina and with silicone, sold under the trade name Solaveil™ CT-12W by Croda,
with TiO2 coated with lauroyl lysine,
with TiO2 coated with C9-C15 fluoroalcohol phosphate and with aluminium hydroxide.

In some embodiments, the disclosure provides a UV absorbing system essentially containing at least one physical ultraviolet (UV)-attenuating material.

In some embodiments, the UV absorbing system has one or more of the following properties:
the physical ultraviolet (UV)-attenuating materials comprise metal oxide(s) such as, for example, oxides of titanium, chromium, zinc, tin, alumina, cerium, and/or iron;
the metal oxide(s) include at least one metal oxide selected from the group consisting of titanium dioxide, zinc oxide, iron oxide, chromium oxide, tin oxide, alumina, cerium oxide, and mixtures thereof;
the physical UV-attenuating material is subjected to a surface treatment agent to improve sensory, performance, and/or compatibility of the compositions disclosed herein, comprising at least one of amino acids, beeswax, fatty acids, fatty acid salts, fatty alcohols, anionic surfactants, lecithin compounds, metal alkoxides, polyethylene, silicones, proteins, alkanolamines, silicon oxides, metal oxides different from the at least one physical ultraviolet (UV)-attenuating material, sodium hexametaphosphate, alumina, glycerol, and mixtures thereof;
the physical UV-attenuating material comprises one or more of zinc oxide and/or titanium dioxide;
the physical UV-attenuating material comprising one or more of zinc oxide or titanium dioxide is not passivated;
the physical UV-attenuating material comprising one or more of zinc oxide or titanium dioxide is passivated;
the physical UV-attenuating material comprises titanium dioxide, which may be treated (coated) or untreated;
the physical UV-attenuating material comprises zinc oxide, which may be treated (coated) or untreated;
the physical UV-attenuating material comprising one or more of treated (coated) zinc oxide, and the zinc oxide is not passivated;
the physical UV-attenuating material comprising one or more of treated (coated) zinc oxide, and the zinc oxide is passivated;
the mean primary particle size of the physical ultraviolet (UV)-attenuating material is from 1 nm to 500 nm, in some embodiments from 5 nm to 250 nm, in some embodiments from 10 nm to 100 nm, and in some embodiments from 20 nm to 50 nm, including all ranges and subranges therebetween such as, for example, 25 nm to 40 nm, 10 nm to 75 nm, and 15 nm to 150 nm;
the coatings can include comprise one or more of the following: hydrated silica, triethoxysilylethyl polydimethylsiloxyethyl hexyl dimethicone, hydrogen dimethicone, triethoxycaprylylsilane, and mixtures thereof; and/or
the coatings can include comprise hydrated silica and/or hydrogen dimethicone.

In some embodiments as exemplified herein, the coatings which comprise triethoxycaprylylsilane (˜0.5-3%), all amounts by weight, based on the weight of the composition.

In some embodiments as exemplified herein, the coatings which comprise triethoxycaprylylsilane (˜0.5-3%), and triethylhexanoin (˜0.5-3.0%), all amounts by weight, based on the weight of the composition.

In some embodiments as exemplified herein, the coatings which comprise triethoxycaprylylsilane (˜1%), and triethylhexanoin (˜1.6-2.4%), all amounts by weight, based on the weight of the composition.

### Physical Ultraviolet (UV)-Attenuating Materials

The mineral sunscreen composition includes one or more physical UV attenuating material.

The physical UV attenuating material used for the present disclosure may be active in the UV-A and/or UV-B region. The physical UV attenuating material may be hydrophilic and/or lipophilic. The physical UV attenuating material is, in some embodiments, insoluble in solvents, such as water, and ethanol commonly used in cosmetics.

It is in some embodiments, desirable that the physical UV attenuating material be in the form of a fine particle such that the mean (primary) particle diameter thereof ranges from 1 nm to 50 nm, and, in some embodiments, 5 nm to 40 nm, and, in some embodiments, 10 nm to 30 nm. The mean (primary) particle size or mean (primary) particle diameter herein is an arithmetic mean diameter. In some embodiments, mean primary particle size of the physical UV attenuating material is from 1 nm to 500 nm, and, in some embodiments, from 5 nm to 250 nm, and, in some embodiments, from 10 nm to 100 nm, and, in some embodiments, from 15 nm to 35 nm, including all ranges and subranges therebetween such as, for example, 25 nm to 40 nm, 10 nm to 75 nm, and 15 nm to 150 nm.

The physical UV attenuating material can be selected from the group consisting of silicon carbide, metal oxides, which may or may not be coated, and mixtures thereof. And in some embodiments, the physical UV attenuating materials are selected from pigments (mean size of the primary particles: generally from 1 nm to 50 nm, and, in some embodiments, from 10 nm to 35 nm) formed of metal oxides, such as, for example, pigments formed of titanium oxide (amorphous or crystalline in the rutile and/or anatase form), iron oxide, zinc oxide, zirconium oxide, or cerium oxide, which are all UV photoprotective agents that are well known per se. And in some embodiments, the physical UV attenuating materials are selected from titanium oxide, zinc oxide, and, in some embodiments, titanium oxide.

The physical UV attenuating material may or may not be coated. The physical UV attenuating material may have one or more coating. The coating may comprise one or more compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes, such as beeswax, (meth)acrylic polymers, organic UV filters, and (per) fluoro compounds. In some embodiments, desirable for the coating to include one or more organic UV filter.

The physical UV attenuating material made of metal oxides may, before their treatment with silicones, have been treated with other surfacing agents, in particular, with cerium oxide, alumina, silica, aluminum compounds, silicon compounds, or their mixtures. The coated physical UV attenuating material may have been prepared by subjecting the physical UV attenuating material to one or more surface treatments of a chemical, electronic, mechanochemical, and/or mechanical nature with any of the compounds as described above, as well as polyethylenes, metal alkoxides (titanium or aluminum alkoxides), metal oxides, sodium hexametaphosphate, and those shown, for example, in Cosmetics & Toiletries, February 1990, Vol. 105, pp. 53-64.

Suitable examples of uncoated zinc oxide include, for example, zinc oxide marketed under the name “Z-COTE”® by BASF, zinc oxide marketed under the name “NanoArcR Zinc Oxide” by the company Nanophase Technologies, zinc oxide marketed under the name “MZ-500”, “MZ-300”, “MZ-200” or “MZ-150” by TAYCA.

Treated (coated) zinc oxide compounds are compounds that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal alkoxides (titanium or aluminum), polyethylene, silicones, hydrated silica, proteins (collagen, elastin), alkanolamines, silicon oxides, triethoxycaprylylsilane, metal oxides or sodium hexametaphosphate.

Examples of suitable coated zinc oxide include but are not limited to zinc oxide coated with polymethylhydrogensiloxane; zinc oxide dispersed in C12-15 alkyl benzonate (INCI: Zinc Oxide (and) C12-15 Alkyl Benzoate (and) Polyhydroxystearic Acid (and) Isostearic Acid), marketed by Croda under the tradename Sovaveil CZ-100; zinc oxide dispersions in C9-12 alkane with a dispersing agent, marketed under the tradename “DAITOPERSION Zn-60VA”® by the company Daito Kasei; ZnO coated with silicone grafted acrylic polymer, dispersed in cyclodimethylsiloxane, marketed under the name “SPD-Z5®” by Shin-Etsu; ZnO coated with hydrated silica, marketed by TAYCA under the name “MZ-500HP”; ZnO coated with hydrated silica, triethoxysilylethyl polydimethylsiloxyethyl hexyl dimethicone and hydrogen dimethicone (H-Me-Si), marketed by TAYCA under the name MZ-510 HPSX; ZnO coated with stearic acid or isostearic acid, such as those marketed by TAYCA under the name “MZ-505T”, “MZY-505EX” or “MZY-304EX”; ZnO coated with silicone oil, such as those marketed by TAYCA under the name “MZX-510HPS”, “MZY-505S”, “MZY-510M3S”, “MZ-505M”, “MZY-303S”, “MZY-303M”, “MZY-203S”, “MZY-210M3S” or “MZY-153S”; ZnO coated with triethoxycaprylylsilane, such as those sold by BASF under the name Z-COTE HP1, or by TAYCA under the name “MZX-508OTS”, “MZY-203OTS” or “MZX-304OTS” or by DSM under the name PARSOL ZX; for example: ZnO marketed under the trademark “Oxide Zinc CS-5” by Toshiba (ZnO coated with polymethylhydrosiloxane); ZnO marketed under the trademark “Nanogard Zinc Oxide FN” by Nanophase Technologies (as a 40% dispersion in Finsolv TN, C12-C15 alkyl benzoate); ZnO marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); ZnO marketed under the trademark “NFD Ultrafine ZnO” by Daikin (ZnO coated with phosphate of perfiuoroalkyl and a copolymer based on perfluoroalkylethyl as a dispersion in cyclopentasiloxane); ZnO marketed under the trademark “SPD-Z1” by Shin-Etsu (ZnO coated with a silicone-grafted acrylic polymer dispersed in cyclodimethylsiloxane); ZnO marketed under the trademark “Escalol Z100” by ISP (alumina-treated ZnO dispersed in an ethylhexyl methoxycinnamate/PVP-hexadecene copolymer/methicone mixture); ZnO marketed under the trademark “Fuji ZnO-SMS-10” by Fuji Pigment (ZnO coated with silica and polymethylsilsesquioxane); and ZnO marketed under the trademark “Nanox Gel TN” by Elementis (ZnO dispersed at 55% in C12-C15 alkyl benzoate with hydroxystearic acid polycondensate); ZnO marketed under the trademark Finex by SAKAI such as FINEX-50LP, FINEX-50S-LP2 and FINEX-30S-LPT (ZnO coated with Hydrogen dimethicone); FINEX-33W (ZnO coated with Hydrated silica), FINEX-52W-LP2 and FINEX-33W-LP2 (ZnO coated with Hydrogen dimethicone and Hydrated silica), FINEX-50-OTS and FINEX-30-OTS (ZnO coated with triethoxycaprylysilane). Preferred coatings may comprise one or more of the following: hydrated silica, triethoxysilylethyl polydimethylsiloxyethyl, hexyl dimethicone and/or hydrogen dimethicone, triethoxycaprylysilane, or a combination thereof.

According to preferred embodiments of the present disclosure, the zinc oxide may be in platelet form, and may be coated or uncoated. Suitable examples of such forms are sold by Croda under the Solaveil (MicNo) name such as Solaveil MXP3, MZP7, MZP8, MZ3-100, MZ3-300 AND MZ7-100. In some embodiments, zinc oxide platelets useful according to the present disclosure (1) have a median specific surface area of more than 25 square meters per gram, and, in some embodiments, greater than 30 square meters per gram, and/or (2) are transparent (that is, >30% transmission at 600 nm). Suitable examples of such platelet forms can also be found in U.S. Pat. No. 11,608,275, the entire contents of which is hereby incorporated by reference in its entirety.

Mention may also be made of TiO₂ pigments doped with at least one transition metal such as iron, zinc or manganese and more particularly manganese. In some embodiments, said doped pigments are in the form of an oily dispersion. The oil present in the oily dispersion is and, in some embodiments, chosen from triglycerides including those of capric/caprylic acids. The oily dispersion of titanium oxide particles may also comprise one or more dispersants, for instance a sorbitan ester, for instance sorbitan isostearate, or a polyoxyalkylenated fatty acid ester of glycerol, for instance Tri-PPG-3 myristyl ether citrate and polyglyceryl-3 polyricinoleate. In some embodiments, the oily dispersion of titanium oxide particles includes at least one dispersant chosen from polyoxyalkylenated fatty acid esters of glycerol. Mention may be made more particularly of the oily dispersion of TiO₂ particles doped with manganese in capric/caprylic acid triglyceride in the presence of Tri-PPG-3 myristyl ether citrate and polyglyceryl-3 polyricinoleate and sorbitan isostearate having the INCI name: titanium dioxide (and) TRI-PPG-3 myristyl ether citrate (and) polyglyceryl-3 ricinoleate (and) sorbitan isostearate, for instance the product sold under the trade name Optisol™ OTP-1 by Croda.

The uncoated titanium oxide pigments are for example sold by Tayca under the trade names MT-500B or MT-600BR, or by Evonik under the name Degussa P 25.

More generally, the coated physical UV attenuating material may be titanium oxides coated: with silica, such as the product “Sun veil” from Ikeda, and “Sunsil TIN 50” from Sunjin Chemical; with silica and with iron oxide, such as the product “Sunveil F” from Ikeda; with silica and with alumina, such as the products “Microtitanium Dioxide MT 500 SA” from Tayca, “Tioveil” from Tioxide, and “Mirasun TiW 60” from Rhodia; with alumina, such as the products “Tipaque TTO-55 (B)” and “Tipaque TTO-55 (A)” from Ishihara, and “UVT 14/4” from Kemira; with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck; with alumina and with aluminum laurate, such as the product “Microtitanium Dioxide MT 100 S” from Tayca; with iron oxide and with iron stearate, such as the product “Microtitanium Dioxide MT 100 F” from Tayca; with zinc oxide and with zinc stearate, such as the product “BR351” from Tayca; with silica and with alumina and treated with a silicone, such as the products “Microtitanium Dioxide MT 600 SAS”, “Microtitanium Dioxide MT 500 SAS”, and “Microtitanium Dioxide MT 100 SAS” from Tayca; with silica, with alumina and with aluminum stearate and treated with a silicone, such as the product “STT-30-DS” from Titan Kogyo; with silica and treated with a silicone, such as the product “UV-Titan X 195” from Kemira; with alumina and treated with a silicone, such as the products “Tipaque TTO-55 (S)” from Ishihara or “UV Titan M 262” from Kemira; with triethanolamine, such as the product “STT-65-S” from Titan Kogyo; with stearic acid, such as the product “Tipaque TTO-55 (C)” from Ishihara; or with sodium hexametaphosphate, such as the product “Microtitanium Dioxide MT 150 W” from Tayca. Other titanium oxide pigments treated with a silicone are, and, in some embodiments, TiO2 treated with octyltrimethylsilane and for which the mean size of the individual particles is from 25 and 40 nm, such as that marketed under the trademark “T 805” by Degussa Silices, TiO2 treated with a polydimethylsiloxane and for which the mean size of the individual particles is 21 nm, such as that marketed under the trademark “70250 Cardre UF TiO2Si3” by Cardre, and anatase/rutile TiO2 treated with a poly dimethylhydrosiloxane and for which the mean size of the individual particles is 25 nm, such as that marketed under the trademark “Microtitanium Dioxide USP Grade Hydrophobic” by Color Techniques.

And in some embodiments, the following coated TiO2 can be used as the coated physical UV attenuating material: Stearic acid (and) Aluminum Hydroxide (and) TiO2, such as the product “MT-100 TV” from Tayca, with a mean primary particle diameter of 15 nm; Dimethicone (and) Stearic Acid (and) Aluminum Hydroxide (and) TiO2, such as the product “S A-TTO-S4” from Miyoshi Kasei, with a mean primary particle diameter of 15 nm; Silica (and) TiO2, such as the product “MT-100 WP” from Tayca, with a mean primary particle diameter of 15 nm; Dimethicone (and) Silica (and) Aluminum Hydroxide (and) TiO2, such as the product “MT-Y02” and “MT-Y-110 M3S” from Tayca, with a mean primary particle diameter of 10 nm; Dimethicone (and) Aluminum Hydroxide (and) TiO2, such as the product “SA-TTO-S3” from Miyoshi Kasei, with a mean primary particle diameter of 15 nm; Dimethicone (and) Alumina (and) TiO2, such as the product “UV TITAN MI 70” from Sachtleben, with a mean primary particle diameter of 15 nm; and Silica (and) Aluminum Hydroxide (and) Alginic Acid (and) TiO2, such as the product “MT-100 AQ” from Tayca, with a mean primary particle diameter of 15 nm. In terms of UV filtering ability, TiO2 coated with one or more organic UV filter is more desirable. For example, Avobenzone (and) Stearic Acid (and) Aluminum Hydroxide (and) TiO2, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”. The uncoated zinc oxide pigments are, for example: those marketed under the trademark “Z-cote” by Sunsmart; those marketed under the trademark “Nanox” by Elementis; and those marketed under the trademark “Nanogard WCD 2025” by Nanophase Technologies. The coated zinc oxide pigments are, for example: those marketed under the trademark “Oxide Zinc CS-5” by Toshiba (ZnO coated with polymethylhydrosiloxane); those marketed under the trademark “Nanogard Zinc Oxide FN” by Nanophase Technologies (as a 40% dispersion in Finsolv TN, C12-C15 alkyl benzoate); those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); those marketed under the trademark “NFD Ultrafine ZnO” by Daikin (ZnO coated with phosphate of perfiuoroalkyl and a copolymer based on perfluoroalkylethyl as a dispersion in cyclopentasiloxane); those marketed under the trademark “SPD-Z1” by Shin-Etsu (ZnO coated with a silicone-grafted acrylic polymer dispersed in cyclodimethylsiloxane); those marketed under the trademark “Escalol Z100” by ISP (alumina-treated ZnO dispersed in an ethylhexyl methoxycinnamate/PVP-hexadecene copolymer/methicone mixture); those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C12-C15 alkyl benzoate with hydroxystearic acid polycondensate). The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”. The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

Mention may also be made of mixtures of metal oxides, in particular, of titanium dioxide and of cerium dioxide, including a mixture of equal weights of titanium dioxide coated with silica and of cerium dioxide coated with silica marketed by Ikeda under the trademark “Sunveil A”, and also a mixture of titanium dioxide and of zinc dioxide coated with alumina, with silica and with silicone, such as the product “M 261” marketed by Kemira, or coated with alumina, with silica and with glycerol, such as the product “M 211” marketed by Kemira. Coated physical UV attenuating materials are desirable, because the UV filtering effects of the physical UV attenuating materials can be enhanced. In addition, the coating(s) may help uniformly or homogeneously disperse the UV filters in the mineral sunscreen composition, according to the present disclosure.

### Other Coated Pigments

Suitable examples of other coated pigments, including those which have amphiphilic property, be selected from:
titanium oxides coated with cetyl phosphate and with silica, such as the product “Eusolex® T-EASY” from Merck;
titanium oxides coated with polyglyceryl-10 oleate and with stearic acid, such as “MTY-200STW” from Tayca;
zinc oxides coated with polyglyceryl-10 oleate and with isostearic acid, such as “MZY-505EXW” from Tayca;
titanium oxides coated with polysorbate 80 and with isostearic acid, such as “MT-10EXW” from Tayca; and
zinc oxides coated with polysorbate 80 and with isostearic acid, such as “MZY-304EXW” from Tayca.

### Organic Sunscreen Agents

If present in the inventive composition, one or more organic UV agents may be selected. Organic UV filter(s) can be hydrophilic or lipophilic. “Hydrophilic organic UV filter” means a water-soluble organic UV filter or a water-dispersible (in colloidal form) organic UV filter. “Lipophilic organic UV filter” means a UV filter which is dissolved or dispersed in colloidal form in a liquid fatty phase.

Organic UV filters can be selected from the following non-exhaustive list of compounds: cinnamic compounds; anthranilate compounds; Para-aminobenzoic acid compounds; salicylic compounds; dibenzoylmethane compounds; camphor compounds; benzophenone compounds; β,β-diphenylacrylate compounds; triazine compounds such as Bis-Ethylhexyloxyphenol Methoxyphenyl Triazine; benzotriazole compounds; benzalmalonate compounds including those mentioned in patent U.S. Pat. No. 5,624,663; benzimidazole derivatives; imidazoline compounds; bis-benzoazolyl compounds as described in patents EP669323 and U.S. Pat. No. 2,463,264; methylene bis-(hydroxyphenyl benzotriazole) compounds as described in applications U.S. Pat. Nos. 5,237,071, 5,166,355, GB2303549, DE 197 26 184 and EP893119; benzoxazole compounds as described in patent applications EP0832642, EP1027883, EP1300137 and DE10162844; polymer filters and silicone filters such as those described in particular in application WO-93/04665; dimers derived from -alkylstyrene such as those described in patent application DE19855649; 4,4-diarylbutadienes compounds as described in applications EP0967200, DE19746654, DE19755649, EP-A-1008586, EP1133980 and EP133981 and mixtures thereof. In some examples, the lipophilic organic UV filters are selected from salicylic compounds, dibenzoylmethane compounds, benzylidene camphor compounds; benzophenone compounds; triazine compounds; benzotriazole compounds; as well as other categories of compounds identified herein; and mixtures thereof.

Specific reference can be made to suitable salicylic compounds including Homosalate (homomentyl salicylate), for example marketed under the trademark “Eusolex HMS” by Rona/EM Industries; and ethylhexyl salicylate, for example marketed under the trademark “Neo Heliopan OS” by Symrise; and glycol salicylate. Other examples of salicylate compounds include phenyl salicylate; dipropyleneglycol salicylate, for example marketed under the trademark “Dipsal” by Scher; and TEA salicylate, for example marketed under the trademark “Neo Heliopan TS” by Symrise.

Examples of suitable β.B-Diphenylacrylate compounds include Octocrylene, for example marketed under the trademark “Uvinul N539” by BASF; and Etocrylene, for example marketed under the trademark “Uvinul N35” by BASF.

Suitable anthranilic compounds can include menthyl anthranilates, for example marketed under the trademark “Neo Heliopan MA” by Symrise.

Examples of dibenzoylmethane compounds include Butyl methoxydibenzoylmethane, for example marketed under the trademark “Parsol 1789” by DSM; and isopropyl dibenzoylmethane.

Suitable cinnamic compounds include Ethylhexyl methoxycinnamate, for example marketed under the trademark “Parsol MCX” by DSM; isopropyl methoxycinnamate; isopropoxy methoxycinnamate; isoamyl methoxycinnamate, for example marketed under the trademark “Neo Heliopan E 1000” by Symrise; cinoxate (2-ethoxyethyl-4-methoxy cinnamate); DEA methoxycinnamate; diisopropyl methylcinnamate; and glyceryl ethylhexanoate dimethoxycinnamate.

Examples of camphor compounds include benzylidenecamphor derivatives: 3-benzylidene camphor, for example marketed under the trademark “Mexoryl SD” by Chimex; 4-methylbenzylidene camphor, for example marketed under the trademark “Eusolex 6300” by Merck; benzylidene camphor sulfonic acid, for example marketed under the trademark “Mexoryl SL” by Noveal; camphor benzalkonium methosulfate, for example marketed under the trademark “Mexoryl SO” by Noveal; terephthalylidene dicamphor sulfonic acid, for example marketed under the trademark “Mexoryl SX” by Noveal; and polyacrylamidomethyl benzylidene camphor, for example marketed under the trademark “Mexoryl SW” by Noveal.

Suitable benzophenone compounds include benzophenone-1 (2,4-dihydroxybenzophenone), such as that marketed under the trademark “Uvinul 400” by BASF; benzophenone-2 (Tetrahydroxybenzophenone), such as that marketed under the trademark “Uvinul D50” by BASF; Benzophenone-3 (2-hydroxy-4-methoxybenzophenone) or oxybenzone, such as that marketed under the trademark “Uvinul M40” by BASF; benzophenone-4 (hydroxymethoxy benzophonene sulfonic acid), such as that marketed under the trademark “Uvinul MS40” by BASF; benzophenone-5 (Sodium hydroxymethoxy benzophenone Sulfonate); benzophenone-6 (dihydroxy dimethoxy benzophenone); such as that marketed under the trademark “Helisorb 11” by Norquay; benzophenone-8, such as that marketed under the trademark “Spectra-Sorb UV-24” by American Cyanamid; benzophenone-9 (Disodium dihydroxy dimethoxy benzophenonedisulfonate), such as that marketed under the trademark “Uvinul DS-49” by BASF; and benzophenone-12, and n-hexyl 2-(4-diethylamino-2-hydroxy benzoyl)benzoate (such as that marketed under the tradename UVINUL A+ by BASF).

Examples of triazine compounds include 4-bis-{[4-(2-ethyl hexyloxy)-2-hydroxy]-phenyl]-6-(4-methoxyphenyl)-1,3,5-triazine (INCI name: Bis-Ethylhexyloxyphenol Methoxyphenyl Triazine (BEMT)), Diethylhexyl butamido triazone, such as that marketed under the trademark “Uvasorb HEB” by Sigma 3V; 2,4,6-tris (dineopentyl 4′-aminobenzalmalonate)-s-triazine, bis-ethylhexyloxyphenol methoxyphenyl triazine, such as that marketed under the trademark “TINOSORB S” by BASF, and ethylhexyl triazone, such as that marketed under the trademark “UVTNUL T150” by BASF.

Suitable benzotriazole compounds include phenylbenzotriazole derivatives: 2-(2H-benzotriazole-2-yl)-6-dodecyl-4-methylpheno, branched and linear; and those described in U.S. Pat. No. 5,240,975.

Suitable benzal malonate compounds include Dineopentyl 4′-methoxybenzalmalonate, and polyorganosiloxane comprising benzalmalonate functional groups, such as polysilicone-15, such as that marketed under the trademark “Parsol SLX” by Hoffmann-LaRoche.

Examples of benzimidazole compounds include, in particular, phenylbenzimidazole derivatives such as phenylbenzimidazole sulfonic acid, such as that marketed in particular under the trademark “Eusolex 232” by Merck, and disodium phenyl dibenzimidazole tetrasulfonate, such as that marketed under the trademark “Neo Heliopan AP” by Symrise.

Suitable imidazoline compounds include Ethylhexyl dimethoxybenzylidene dioxoimidazoline propionate.

Examples of bis-benzoazolyl compounds include the compounds described in EP-669,323 and U.S. Pat. No. 2,463,264.

Suitable para-aminobenzoic acid compounds include PABA (p-aminobenzoic acid), ethyl PABA, Ethyl dihydroxypropyl PABA, pentyl dimethyl PABA, ethylhexyl dimethyl PABA, such as that marketed under the trademark “Escalol 507” by ISP, glyceryl PABA, and PEG-25 PABA, such as that marketed under the trademark “Uvinul P25” by BASF.

Suitable methylene bis-(hydroxyphenylbenzotriazol) compounds include 2,2′-methylenebis [6-(2H-benzotriazol-2-yl)-4-methyl-phenol], such as that marketed under the trademark “Mixxim BB/200” by Fairmount Chemical, 2,2′-methylenebis [6-(2H-benzotriazol-2-yl)-4-(1,1,3,3-tetramethylbutyl) phenol], such as that marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, such as that marketed under the trademark “Silatrizole” by Rhodia Chimie or- “Mexoryl XL” by L'Oréal.

Examples of benzoxazole compounds include 2,4-bis [5-l(dimethylpropyl)benzoxazol-2-yl-(4-phenyl)imino]-6-(2-ethylhexyl)imino-1,3,5-triazine, such as that marketed under the trademark of Uvasorb K2A by Sigma 3V.

Suitable examples of screening polymers and screening silicones include the silicones described in WO 93/04665.

Suitable dimers derived from a-alkylstyrene include the dimers described in DE-19855649.

Examples of 4,4-Diarylbutadiene compounds include 1,1-dicarboxy (2,2′-dimethylpropyl)-4,4-diphenylbutadiene.

### Emulsifier (Emulsifier) System

In various embodiments, the mineral sunscreen composition includes at least one low HLB and at least one high HLB emulsifier, wherein at least one emulsifier has a low HLB that is equal to or less than 6 (for example, about 5) and at least one emulsifier has a high HLB that is greater than 8 (for example, about 15).

In various embodiments, the mineral sunscreen composition includes two or more emulsifiers, and in such embodiments, at least one emulsifier has a low HLB that is equal to or less than 6 (for example, about 5), and in the range from about 1-8, and at least one emulsifier has a high HLB that is greater than 9 (for example, about 13-15) and in the range from about 9-25. In accordance with such embodiments, at least one emulsifier has an HLB in the range from about 1 to about 8, for example from about 1, 2, 3, 4, 5, or 6, and at least one emulsifier has an HLB in the range from about 9 to about 25, for example, from about 9, 10, 11, 12, 13, 14, 15, 16, 17, 18, 19, 20, 21, 22, 23, 24 or 25.

In some embodiments, the mineral sunscreen composition includes one or more of glyceryl stearate (HLB˜3.8), steareth-2 (HLB˜5) and ceteth-2 (HLB˜5), and one or more of polysorbate 20 (HLB˜16.7), steareth-20 (HLB˜15), and decyl glucoside (HLB˜13-15).

In some embodiments, the mineral sunscreen composition includes glyceryl stearate (HLB˜3.8), steareth-2 (HLB˜5), polysorbate 20 (HLB˜16.7), and steareth-20 (HLB˜15).

In some embodiments as exemplified herein, the mineral sunscreen composition includes glyceryl stearate (HLB˜3.8: ˜0.2-1.5%), steareth-2 (HLB˜5; ˜0.25-1.25%), polysorbate 20 (HLB˜16.7; ˜0.25-3.0%), and steareth-20 (HLB˜15: ˜0.5-3.0%), all amounts by weight, based on the weight of the composition.

In some embodiments as exemplified herein, the mineral sunscreen composition includes glyceryl stearate (˜0.7%), steareth-2 (˜0.6%), polysorbate 20 (˜0.7%), and steareth-20 (˜1%), all amounts by weight, based on the weight of the composition.

In some embodiments, each emulsifier is present in the mineral sunscreen composition in an amount ranging from about 0.01% to about 15% by weight relative to the weight of the mineral sunscreen composition. In some embodiments, each emulsifier is present in the mineral sunscreen composition in an amount ranging from about 0.05% to about 3.5%, or from about 0.3% to about 1.5%, or from about 0.5% to about 0.9%, by weight of the mineral sunscreen composition, including increments and ranges therein and there between, based upon the total weight of the mineral sunscreen composition.

In some embodiments, a combination of emulsifier is present in the mineral sunscreen composition from about 1% to about 5% by weight, or from about 2% to about 4%, by weight, or any suitable combination, sub-combination, range, or sub-range thereof by weight, based on the weight of the mineral sunscreen composition.

Thus, one or a combination of emulsifiers, is present, by weight, based on the weight of the mineral sunscreen composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, to about 15 percent, including increments and ranges therein and there between, based upon the total weight of the mineral sunscreen composition.

### Aqueous Phase Hydrophobically-Modified Anionic Polymeric Thickener

In various embodiments, the mineral sunscreen composition includes at least one aqueous phase hydrophobically-modified anionic polymeric thickener comprising carboxylic acid residues.

In some embodiments, the aqueous phase hydrophobically-modified polymeric thickener further functions as a polymeric emulsifier.

In some embodiments, the aqueous phase hydrophobically-modified anionic polymeric thickener comprises acrylates/C10-30 alkyl acrylate crosspolymer which excludes the commercial material Carbopol® Ultrez 20 Polymer® (INCI Name: Acrylates/C10-30 Alkyl Acrylate Crosspolymer).

In some embodiments, the aqueous phase hydrophobically-modified anionic polymeric thickener comprises a cross-linked acrylate copolymer. Examples of cross-linked acrylate copolymer include, but are not limited to, acrylates/C10-30 alkyl acrylate crosspolymer.

In some embodiments as exemplified herein, the mineral sunscreen composition includes acrylates/C10-30 alkyl acrylate crosspolymer (˜0.1-0.5%), all amounts by weight, based on the weight of the composition.

In some embodiments as exemplified herein, the mineral sunscreen composition includes acrylates/C10-30 alkyl acrylate crosspolymer (˜0.3%), all amounts by weight, based on the weight of the composition.

In some embodiments, the aqueous phase hydrophobically-modified anionic polymeric thickener also has emulsifying properties.

The at least one aqueous phase hydrophobically-modified anionic polymeric thickener is present at a concentration from about 0.05% to about 5%, or from about 0.1% to about 2.5%, or from about 0.1% to about 1.5%, or from about 0.2% to about 1%, or from about 0.25% to about 0.5%, or any suitable combination, sub-combination, range, or sub-range thereof by weight, based on the weight of the mineral sunscreen composition.

Thus, in various embodiments the least one aqueous phase hydrophobically-modified anionic polymeric thickener is present in the mineral sunscreen composition from 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.5, 1.5, 1.6, 1.7, 1.8, 1.9, 2.0, 2.1, 2.2, 2.3, 2.4, 2.5 percent, including increments and ranges therein and there between, based upon the total weight of the mineral sunscreen composition.

### Polymeric Dispersant

various embodiments, the mineral sunscreen compositions may contain at least one polymeric dispersant. In some embodiments, the polymeric dispersant comprises polyhydroxystearic acid.

In some embodiments, the polymeric dispersant comprises polyhydroxystearic acid (˜1-5%).

When present, the at least one polymeric dispersant is present at a concentration from about 1% to 25%, or from about 1% to 10%, or from about 1% to 5%, or from about 1.5% to 2.5%, or from about 2% to about 2.5, or any suitable combination, sub-combination, range, or sub-range thereof by weight, based on the weight of the mineral sunscreen composition.

Thus, in various embodiments the least one polymeric dispersant is present in the mineral sunscreen composition from 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0 to 25.0 percent, including increments and ranges therein and there between, based upon the total weight of the mineral sunscreen composition.

### Rheology Modifiers

In various embodiments, the mineral sunscreen compositions may contain at least one rheology modifier. In some instances, certain rheology modifiers can be gelling agents.

In some embodiments, when present, the at least one rheology modifier is selected from the group consisting of xanthan gum, cellulose gum, biosaccharide gum, carrageenan, gellan gum, or associative thickeners such as like Sepinov EMT10™ or Sepigel™, Aristoflex™, and combinations thereof.

In some embodiments, the at least one rheology modifier includes xanthan gum (˜0.1-0.5%), all amounts by weight, based on the weight of the composition.

Broadly, the rheology modifier(s) that may be useful in the practice of the present invention include those conventionally used in cosmetics such as polymers of natural origin and synthetic polymers. Rheology modifiers are employed in the mineral sunscreen compositions of the present invention when it is desired to adjust the viscosity or thickness of the mineral sunscreen compositions or to achieve a particular composition texture.

Representative rheology-modifying agents that may be include nonionic, anionic, cationic, and amphoteric polymers, and other rheology modifiers such as cellulose-based thickeners (e.g., hydroxyethyl cellulose, hydroxypropyl cellulose, carboxymethylcellulose, cationic cellulose ether derivatives, quaternized cellulose derivatives, etc.), guar gum and its derivatives (e.g., hydroxypropyl guar, cationic guar derivatives, etc.), gums such as gums of microbial origin (e.g., xanthan gum, scleroglucan gum, etc.), and gums derived from plant exudates (e.g., gum arabic, ghatti gum, karaya gum, gum tragacanth, carrageenan gum, agar gum and carob gum), pectins, alginates, and starches, crosslinked homopolymers of acrylic acid or of acrylamidopropane-sulfonic acid, associative polymers, non-associative thickening polymers, and water-soluble thickening polymers.

Rheology modifiers include polymers chosen from nonionic, anionic, cationic and amphoteric amphiphilic polymers. The rheology-modifying agents may also be chosen from associative celluloses include quaternized cationic celluloses and quaternized cationic hydroxyethyl celluloses modified by groups containing at least one hydrophobic chain, such as alkyl, arylalkyl or alkylaryl groups containing at least 8 carbon atoms, and mixtures thereof. The alkyl radicals carried by the above quaternized celluloses or hydroxyethyl celluloses may, in various embodiments, comprise from 8 to 30 carbon atoms. The aryl radicals may, for example, denote the phenyl, benzyl, naphthyl or anthryl groups. Representative examples of quaternized alkylhydroxy-ethylcelluloses containing a C8-C30 hydrophobic chain include the products Quatrisoft LM 200R, Quatrisoft LM-X 529-18-AR), Quatrisoft LM-X 529-18BR (C12 alkyl) and Quatrisoft LM-X 529-8R (Ci8 alkyl) sold by Amerchol and the products Crodacel QMR, Crodacel QLR (C12 alkyl) and Crodacel QSR (Ci8 alkyl) sold by Croda.

Representative examples of nonionic cellulose derivatives include hydroxyethy 1 celluloses modified by groups comprising at least one hydrophobic chain, such as alkyl, arylalkyl or alkylaryl groups, or their blends, and in which the alkyl groups are, for example, C8-C22 alkyl groups, such as the product Natrosol Plus Grade 330 CSR (C16 alkyls) sold by Aqualon or the product Bermocoll EHM 100R sold by Berol Nobel. Representative examples of cellulose derivatives modified by alkylphenyl polyalkylene glycol ether groups include the product Amercell Polymer HM-1500R sold by Amerchol.

Rheology modifiers may be selected from Sepinov EMT10™ or Sepigel™, Aristoflex™, or combinations thereof.

The at least one rheology modifier may be present in an amount ranging from about 0.01% to about 10% by weight, in some embodiments, from about 0.05% to about 5% by weight, or from about 0.1% to about 0.8% by weight, or from about 0.15% to about 0.3, or any suitable combination, sub-combination, range, or sub-range thereof by weight, based on the weight of the mineral sunscreen composition.

Thus, in various embodiments, the at least one rheology modifier may be present in the mineral sunscreen composition from 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, to 10.0 percent, including increments and ranges therein and there between, based upon the total weight of the mineral sunscreen composition.

### Antioxidants

In various embodiments, the mineral sunscreen composition may include at least one SPF boosting antioxidant or a combination of SPF boosting antioxidants.

In some embodiments, the at least one SPF boosting antioxidant is selected from the group consisting of diethylhexyl syringylidenemalonate, ethylhexyl methoxycrylene, and a combination thereof.

In some embodiments, the at least one SPF boosting antioxidant includes diethylhexyl syringylidenemalonate and ethylhexyl methoxycrylene.

In some embodiments as exemplified herein, the at least one SPF boosting antioxidant includes diethylhexyl syringylidenemalonate (˜0.1-1.0%) and ethylhexyl methoxycrylene (˜1.0-3.0%), all amounts by weight based on the weight of the composition.

In some embodiments as exemplified herein, the at least one SPF boosting antioxidant includes diethylhexyl syringylidenemalonate (˜0.45%) and ethylhexyl methoxycrylene (˜2%), all amounts by weight based on the weight of the composition.

In some embodiments, the at least one SPF boosting antioxidant may be selected from Butyloctyl Salicylate, ethylhexyl methoxycrylene, styrene/acrylates copolymer (such as the product sold under the tradename SUNSPHERES™), Ethylenediamine/Stearyl Dimer Dilinoleate Copolymer, Dimethicone and Acrylates/Dimethicone Copolymer, silicone polymer comprising dimethicone (and) dimethicone/vinyl dimethicone copolymer, the UVA booster Solastay, and combinations thereof. In some particular embodiments, the SPF boosting antioxidant comprises one or more of Butyloctyl Salicylate and styrene/acrylates copolymer.

The at least one SPF boosting antioxidant may be at a concentration from about 0.05% to 10%, in some embodiments, from about 0.1% to 5%, or from about 0.4% to 0.5%, or from about 1.5% to 2.5%, or any suitable combination, sub-combination, range, or sub-range thereof by weight, based on the weight of the mineral sunscreen composition.

Thus, in various embodiments at least one SPF boosting antioxidant may be present in the mineral sunscreen composition in a weight percent amount from 0.1, 0.2, 0.3, 0.4, 0.5, 0.6, 0.7, 0.8, 0.9, 1.0, 2.0, 3.0, 4.0, to 5.0 percent, including increments and ranges therein and there between, based upon the total weight of the mineral sunscreen composition.

### Solvent Systems for Water and Oil Phases

In various embodiments, the mineral sunscreen composition according to the disclosure includes and a solvent system that water and oil phase solvents, wherein solvents in the water phase are selected from water and water-based solvents, and wherein the oil phase solvents includes one or a combination of emollients and silicones. Accordingly in various embodiments, the solvent system comprises one or more oils/fatty compounds/emollients and silicones in the oily phase and water in the aqueous phase.

In some embodiments, the solvent system includes water, and one or more of glycerin and propanediol, and includes in the oily phase an oil/fatty compound blend selected from the group consisting of caprylic/capric triglyceride, cetearyl isononanoate, cetearyl alcohol, dicaprylyl carbonate, isohexadecane, at least one C15-19 alkane, dimethicone and combinations thereof.

In some embodiments, the solvent system includes in the water phase at least water, glycerin and propanediol, and includes in the oily phase an oil/fatty compound/emollient blend comprising caprylic/capric triglyceride, cetearyl isononanoate, cetearyl alcohol, dicaprylyl carbonate, isohexadecane, at least one C15-19 alkane, and dimethicone.

In some embodiments as exemplified herein, the solvent system includes in the water phase at least water (˜34-40%), glycerin (˜3-10%) and propanediol (˜3-10%), and includes in the oily phase an oil/fatty compound/emollient blend comprising caprylic/capric triglyceride (˜0.01-0.1%), cetearyl isononanoate (˜0.5-5.0%), cetearyl alcohol (˜0.2-1.5%), dicaprylyl carbonate (˜0.5-7.0%), isohexadecane (˜1.0-5.0%), at least one C15-19 alkane (˜0.5-10%), and dimethicone (˜0.5-20%), all amounts by weight, based on the weight of the composition.

In some embodiments as exemplified herein, the solvent system includes in the water phase at least water (˜36%), glycerin (˜5%) and propanediol (˜3%), and includes in the oily phase an oil/fatty compound/emollient blend comprising caprylic/capric triglyceride (˜0.05%), cetearyl isononanoate (˜2.5%), cetearyl alcohol (˜0.6%), dicaprylyl carbonate (˜4-5%), isohexadecane (˜1.6-2.4%), at least one C15-19 alkane (˜6.5-7%), and dimethicone (˜1-2%), all amounts by weight, based on the weight of the composition.

In various embodiments, the amount of each solvent or combination thereof present in the mineral sunscreen compositions can range from about 0.01% to about 70% based on the weight of the mineral sunscreen composition. In some embodiments, the solvent system includes water present from about 10% to about 70%, or from about 20% to about 50%, or about 30% to about 40%. In some embodiments, the solvent system includes one or a combination of oils/oil/fatty compounds that can include silicone and non-silicone compounds, each of which can be present in a range from about 0.05% to about 25%, and in some embodiments in combination up to about 60%. In various embodiments, the solvent system includes components present from about 0.01% to about 70%, or from about 0.05% to about 50%, or from about 0.1% to about 45%, or from about 0.1% to about 20%, or from about 0.1% to about 10%, or from about 0.5% to about 5% or any suitable combination, sub-combination, range, or sub-range thereof by weight, based on the weight of the mineral sunscreen composition. One of ordinary skill in the art, however, will appreciate that other ranges are within the scope of the invention.

Thus, any one solvent may be present, by weight, based on the total weight of the mineral sunscreen composition, is from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, to about 70 percent, including increments and ranges therein and there between, based upon the total weight of the mineral sunscreen composition.

In various embodiments, the mineral sunscreen composition according to the disclosure includes in the oil phase solvent system one or more oils selected from hydrocarbons, silicones, fatty alcohols, glycols, and vegetable oils. The oil may include one or a combination of polar and non-polar oil. In some embodiments, the oil may be chosen from hydrocarbon-based oils from plants or of plant origin, mineral oil, ester oils, fatty alcohols containing from 12 to 26 carbon atoms, fatty acids containing from 12 to 26 carbon atoms and vinylpyrrolidone copolymers, and combinations thereof.

The term “silicone oil” relates to oil comprising at least one silicon atom, and especially at least one Si—O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil, or a synthetic hydrocarbon-based oil. Further, suitable oil may be a mineral hydrocarbon-based oil, a plant hydrocarbon-based oil, or a synthetic hydrocarbon-based oil.

### Hydrocarbon-Based Oils

The mineral sunscreen composition may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the mineral sunscreen composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 40 squalane;
(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsoly TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di (linear C12-C13 alkyl) tartrates, such as those sold under the name Cosmacol ETI® by Enichem Augusta Industriale, and also di (linear C14-C15 alkyl) tartrates, such as those sold under the name Cosmacol ETLR by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid, or linolenic acid;
(vii) carbonates, such as dicaprylyl carbonate, such as the product sold under the name Cetiol CC™ by Cognis;
(viii) fatty amides, such as isopropyl N-lauroyl sarcosinate, such as the product sold under the trade name Eldew SL 205™ from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Hydrocarbon-based oils may be glyceride triesters and in particular to caprylic/capric acid triglycerides, synthetic esters and in particular isononyl isononanoate, oleyl erucate, C12-C15 alkyl benzoate, 2-ethylphenyl benzoate and fatty alcohols, such as octyldodecanol. As volatile hydrocarbon-based oils, mention is made of hydrocarbon-based oils containing from 8 to 16 carbon atoms and in particular of branched C8-C16 alkanes, such as C8-C16 isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C C8-C16 esters, and isohexyl neopentanoate.

In some embodiments, the mineral sunscreen composition may comprise one or more oils such as from those described herein above, and from oils that may be selected from branched or linear, liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some embodiments, suitable liquid alkanes that may be used according to the disclosure include hydrocarbon-based oils containing from 8 to 16 carbon atoms, and especially branched C8-C16 alkanes such as C8-C16 isoalkanes.

In some embodiments, the mineral sunscreen composition may comprise one or more oils selected from polar oil/fatty compounds selected from esters, triglycerides, ethers, carbonates, alcohols, oils, butters, fatty acids, and their combinations thereof. In various embodiments, the polar oil/fatty compounds may be selected from those with a molecular weight of 400 g/mol or less. More, generally, the polar oil/fatty compound may have a molecular weight in the range from about 50 g/mol % to about 350 g/mol.

In some embodiments, the mineral sunscreen composition may comprise polar oil/fatty compounds that include those derived from C12-C50 fatty acids, and, in some embodiments, C16-C22 saturated fatty acids, and monohydric alcohols. In some embodiments, such esters may be chosen from isopropyl myristate, methyl palmitate, isopropyl laurate, isopropyl palmitate, ethylhexyl palmitate, ethylhexyl laurate, ethylhexyl oleate, ethylhexyl isononanoate, myristyl myristate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

### Silicone Oils

The mineral sunscreen composition may comprise one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the mineral sunscreen composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Other examples of silicone oils that may be mentioned include volatile linear or cyclic silicone oils, especially those with a viscosity 8 centistokes (8×106 m2/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

### Fluoro Oils

The mineral sunscreen composition may comprise one or more fluoro oils. For example, the one or more fluoro oil may be selected from the group consisting of perfluoromethylcyclopentane, perfluoro-1,3-dimethylcyclohexane, dodecafluoropentane, tetradecafluorohexane, bromoperfluorooctyl, nonafluoromethoxybutane, nonafluoroethoxyisobutane and 4-trifluoromethylperfluoromorpholine. Volatile fluoro oils, such as nonafluoromethoxybutane, decafluoropentane, tetradecafluorohexane, dodecafluoropentane, may also be used.

### Cosmetic Waxes

In accordance with the disclosure, optionally one or more wax may be present in the mineral sunscreen composition, the wax selected from natural and synthetic waxes. Natural waxes can include animal tallow, bayberry wax, beeswax, grapefruit wax, orange peel wax, palm wax, rice bran wax, sumac wax, sunflower wax, soy wax, polyhydroxystearic acid, and combinations thereof.

In some embodiments, when present, a wax may be selected from Oryza sativa cera (rice bran wax), candelilla wax, sunflower seed wax, carnauba wax, polyhydroxystearic acid, and combinations thereof. In a particular embodiment, the mineral sunscreen composition comprises rice bran wax.

Suitable examples of waxes include, but are not limited to, hydrocarbon waxes, silicone waxes, and/or fluoro waxes, and can be of plant, animal, mineral and/or synthetic origin. By way of examples of hydrocarbon waxes, mention may be made of beeswax, lanolin wax or Chinese insect wax, rice bran wax, carnauba wax, candelilla wax, ouricury wax, esparto grass wax, cork fibre wax, sugar cane wax, Japan wax, Berry wax, shellac wax and sumac wax, Helianthus annuus (sunflower) seed wax, montan wax, microcrystalline waxes, paraffins, ozokerite, polyethylene waxes, polymethylene waxes, waxes obtained by Fischer-Tropsch synthesis and waxy copolymers, and also esters thereof. Mention may in particular be made of beeswax, for example the product sold under the name White beeswax BR G889 by Koster Keunen, carnauba wax, for example sold under the name Cerauba T1 Bio or Cerauba T3 by Baerlocher or under the name Mexoryl SAP by Noveal, Helianthus annuus (sunflower) wax sold under the name Sunflower Wax by Koster Keunen, ORYZA SATIVA (RICE) BRAN WAX, for example sold under the name NC 1710 by Cera Nica Noda, or a mixture thereof.

In accordance with the various embodiments, the one or wax, when present, is present in the mineral sunscreen composition at a concentration, by weight, of between about 0.1% to about 10%, or from about 0.2% to about 4%, or from about 0.5% to about 3%, or from about 1% to about 2%, or any suitable combination, sub-combination, range, or sub-range thereof by weight, based on the weight of the mineral sunscreen composition.

In some embodiments, the mineral sunscreen composition comprises a more than one way, each one present in the mineral sunscreen composition at a concentration, by weight, based on the total weight of mineral sunscreen composition, in the range from about 0.1% to about 10%, or any suitable combination, sub-combination, range, or sub-range thereof by weight, based on the weight of the mineral sunscreen composition.

Thus, when present, the wax is present by weight, based on the total weight of the mineral sunscreen composition from about 0.10, 0.20, 0.30, 0.40, 0.50, 0.60, 0.70, 0.80, 0.90, 1.0, 2, 3, 4, 5, 6, 7, 8, 9, to about 10 weight percent, including increments and ranges therein and there between.

### Hydrating Agents

In accordance with the disclosure, in some embodiments, one or more hydrating agents or humectants may be present in the mineral sunscreen composition. The hydrating agent present in the mineral sunscreen composition, according to the disclosure, includes, but is not limited to, one or more of polyols, including, for example, glycerin, glycerol, glycols, such as caprylyl glycol, butylene glycol, propylene glycol, isoprene glycol, dipropylene glycol, hexylene glycol and polyethylene glycols, monoethylene glycol, diethylene glycol, triethylene glycol, diethylene glycol, hexylene glycol, glycol ethers such as monopropylene, dipropylene and tripropylene glycol alkyl (C1-C4) ethers, squalane, triacetin, sugars, such as glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, and seaweed extract.

In some embodiments, the mineral sunscreen composition includes a hydrating agent selected from one or a combination of glycerin present at about 4% and propanediol present at about 1%.

In various embodiments, the amount of hydrating agent present in the mineral sunscreen composition can range from about 1% to about 25%, or from about 2% to about 20%, or from about 3% to about 5% or any suitable combination, sub-combination, range, or sub-range thereof by weight, based on the weight of the mineral sunscreen composition. One of ordinary skill in the art, however, will appreciate that other ranges are within the scope of the invention.

Thus, any one of or a combination of hydrating agent may be present, by weight, based on the total weight of the mineral sunscreen composition, is from about 1, 2, 3, 4, 5, 6, 7, 8, 9, 10, 11, 12, 13, 14, 15, 16, 17, 18, 19, 20, 21, 22, 23, 24, to about 25 percent, including increments and ranges therein and there between, based upon the total weight of the mineral sunscreen composition.

### Powders/Particles

The mineral sunscreen composition according to the disclosure may optionally comprise at least one or a combination of powders/particles for modifying sensory feel. In some particular embodiments, the powders may be selected from the group consisting of aluminum stearate, alumina, silica, cellulose, boron nitride, perlite, aluminium starch, octenyl succinate and combinations thereof.

In some embodiments, the at least one powder includes cellulose (˜0.5-5.0%), all amounts by weight, based on the weight of the composition.

In some embodiments, the at least one powder includes cellulose (˜1.7%), all amounts by weight, based on the weight of the composition.

The at least one powder, when present, may be at a concentration from about 0.05% to 15%, in some embodiments, from about 0.1% to 10%, or from about 1% to 5%, or any suitable combination, sub-combination, range, or sub-range thereof by weight, based on the weight of the mineral sunscreen composition.

Thus, in various embodiments, a powder, when present, may be present in the mineral sunscreen composition in a weight percent amount from 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, to 15.0 percent including increments and ranges therein and there between, based upon the total weight of the mineral sunscreen composition.

### Organic UV Filters

In some embodiments, the mineral sunscreen composition may include one or more organic UV filters. In some embodiments, the mineral sunscreen composition expressly excludes organic UV filters. Organic UV filter used for the present disclosure may be active in the UV-A and/or UV-B region. The organic UV filter may be hydrophilic and/or lipophilic. The organic UV filter may be solid or liquid. The terms “solid” and “liquid” mean solid and liquid, respectively, at 25° C. under 1 atm.

An organic UV filter can be selected from the group consisting of anthranilic compounds; dibenzoylmethane compounds; cinnamic compounds; salicylic compounds; camphor compounds; benzophenone compounds; ÿ,ÿ-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

Mention may be made, as examples of the organic UV filter(s), of those denoted below under their INCI names, and mixtures thereof. Anthranilic compounds: Menthyl anthranilate, marketed under the trademark “Neo Heliopan MA” by Haarmann and Reimer. Dibenzoylmethane compounds: Butyl methoxydibenzoylmethane, marketed, in particular, under the trademark “Parsol 1789” by Hoffmann-La Roche; and isopropyl dibenzoylmethane. Cinnamic compounds: Ethylhexyl methoxycinnamate, marketed, in particular, under the trademark “Parsol MCX” by Hoffmann-La Roche; isopropyl methoxycinnamate; isopropoxy methoxycinnamate; isoamyl methoxycinnamate, marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate. Salicylic compounds: Homosalate (homomentyl salicylate), marketed under the trademark “Eusolex HMS” by Rona/EM Industries; ethylhexyl salicylate, marketed under the trademark “Neo Heliopan OS” by Haarmann and Reimer; glycol salicylate; butyloctyl salicylate; phenyl salicylate; dipropyleneglycol salicylate, marketed under the trademark “Dipsal” by Scher; and TEA salicylate, marketed under the trademark “Neo Heliopan TS” by Haarmann and Reimer. Camphor compounds, in particular, benzylidenecamphor derivatives: 3-benzylidene camphor, manufactured under the trademark “Mexoryl SD” by Chimex; 4-methylbenzylidene camphor, marketed under the trademark “Eusolex 6300” by Merck; benzylidene camphor sulfonic acid, manufactured under the trademark “Mexoryl SL” by Chimex; camphor benzalkonium methosulfate, manufactured under the trademark “Mexoryl SO” by Chimex; terephthalylidene dicamphor sulfonic acid, manufactured under the trademark “Mexoryl SX” by Chimex; and polyacrylamidomethyl benzylidene camphor, manufactured under the trademark “Mexoryl SW” by Chimex. Benzophenone compounds: Benzophenone-1 (2,4-dihydroxy benzophenone), marketed under the trademark “Uvinul 400” by BASF; benzophenone-2 (Tetrahydroxy benzophenone), marketed under the trademark “Uvinul D50” by BASF; Benzophenone-3 (2-hydroxy-4-methoxybenzophenone) or oxybenzone, marketed under the trademark “Uvinul M40” by BASF; benzophenone-4 (hydroxymethoxy benzophonene sulfonic acid), marketed under the trademark “Uvinul MS40” by BASF; benzophenone-5 (Sodium hydroxymethoxy benzophenone Sulfonate); benzophenone-6 (dihydroxy dimethoxy benzophenone); marketed under the trademark “Helisorb 11” by Norquay; benzophenone-8, marketed under the trademark “Spectra-Sorb UV-24” by American Cyanamid; benzophenone-9 (Disodium dihydroxy dimethoxy benzophenonedisulfonate), marketed under the trademark “Uvinul DS-49” by BASF; and benzophenone-12, and n-hexyl 2-(4-diethylamino-2-hydroxy benzoyl)benzoate (UVINUL A+ by BASF). ÿ,ÿ-Diphenylacrylate compounds; Octocrylene, marketed in particular under the trademark “Uvinul N539” by BASF; and Etocrylene, marketed in particular under the trademark “Uvinul N35” by BASF. Triazine compounds: Diethylhexyl butamido triazone, marketed under the trademark “Uvasorb HEB” by Sigma 3V; 2,4,6-tris (dineopentyl 4′-aminobenzalmalonate)-s-triazine, bis-ethylhexyloxyphenol methoxyphenyl triazine marketed under the trademark «TINOSORB S» by CIBA GEIGY, and ethylhexyl triazone marketed under the trademark «UVTNUL T150» by BASF. Benzotriazole compounds, in particular, phenylbenzotriazole derivatives: 2-(2H-benzotriazole-2-yl)-6-dodecyl-4-methylpheno, branched and linear; and those described in U.S. Pat. No. 5,240,975. Benzalmalonate compounds: Dineopentyl 4′-methoxybenzalmalonate, and polyorganosiloxane comprising benzalmalonate functional groups, such as polysilicone-15, marketed under the trademark “Parsol SLX” by Hoffmann-LaRoche. Benzimidazole compounds, in particular, phenylbenzimidazole derivatives: Phenylbenzimidazole sulfonic acid, marketed in particular under the trademark “Eusolex 232” by Merck, and disodium phenyl dibenzimidazole tetrasulfonate, marketed under the trademark “Neo Heliopan AP” by Haarmann and Reimer. Imidazoline compounds: Ethylhexyl dimethoxybenzylidene dioxoimidazoline propionate. Bis-benzoazolyl compounds: The derivatives as described in EP-669,323 and U.S. Pat. No. 2,463,264. Para-aminobenzoic acid compounds: PABA (p-aminobenzoic acid), ethyl PABA, Ethyl dihydroxypropyl PABA, pentyl dimethyl PABA, ethylhexyl dimethyl PABA, marketed, in particular, under the trademark “Escalol 507” by ISP, glyceryl PABA, and PEG-25 PABA, marketed under the trademark “Uvinul P25” by BASF. Methylene bis-(hydroxyphenylbenzotriazol) compounds, such as 2,2′-methylenebis [6-(2H-benzotriazol-2-yl)-4-methyl-phenol] marketed in the solid form under the trademark “Mixxim BB/200” by Fairmount Chemical, 2,2′-methylenebis [6-(2H-benzotriazol-2-yl)-4-(1,1,3,3-tetramethylbutyl) phenol] marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, marketed under the trademark “Silatrizole” by Rhodia Chimie or- “Mexoryl XL” by L'Oreal. Benzoxazole compounds: 2,4-bis [5-1 (dimethylpropyl)benzoxazol-2-yl-(4-phenyl)imino]-6-(2-ethylhexyl)imino-1,3,5-triazine, marketed under the trademark of Uvasorb K2A by Sigma 3V. Screening polymers and screening silicones: The silicones described in WO 93/04665. Dimers derived from a-alkylstyrene: The dimers described in DE-19855649. 4,4-Diarylbutadiene compounds: 1,1-dicarboxy (2,2′-dimethylpropyl)-4,4-diphenylbutadiene.

Organic UV filter(s) may be selected from the group consisting of: butyl methoxydibenzoylmethane, ethylhexyl methoxycinnamate, homosalate, ethylhexyl salicylate, phenylbenzimidazole sulfonic acid, benzophenone-3, benzophenone-4, benzophenone-5, n-hexyl 2-(4-diethylamino-2-hydroxy benzoyl)benzoate, l,r-(1,4-piperazinediyl)bis[1-[2-[4-(diethylamino)-2-hydroxybenzoyl] phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]-disiloxanyl}propyl) amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxy benzalmalonate, 1,1-dicarboxy(2,2′-dimethylpropyl)-4,4-diphenylbutadiene, 2,4-bis[5-1(dimethylpropyl)benzoxazol-2-yl-(4-phenyl)imino]-6-(2-ethylhexyl)imino-1,3,5-triazine, camphor benzylkonium methosulfate, and mixtures thereof.

### Optional Additives

The mineral sunscreen compositions can also comprise one or more additive used in the cosmetics field which does not adversely affect the properties of the mineral sunscreen compositions according to the invention.

In some embodiments, the mineral sunscreen composition comprises one or more additives selected from the group consisting of skin conditioners, hydrating agents, skin actives, preservatives, antimicrobials, powders, fillers, and combinations thereof.

In some embodiments, the mineral sunscreen composition comprises one or more additives selected from the group consisting of tocopherol, niacinamide, hydroxyacetophenone, citric acid, chlorphenesin, caprylyl glycol, aluminum stearate, alumina, silica, and combinations thereof.

In some embodiments, the mineral sunscreen composition comprises, tocopherol, niacinamide, hydroxyacetophenone, citric acid, chlorphenesin, caprylyl glycol, aluminum stearate, alumina, and silica.

In some embodiments as exemplified herein, the mineral sunscreen composition comprises tocopherol (˜0.1-1.5%), niacinamide (˜0.5-5.0%), hydroxyacetophenone (˜0.1-10%), citric acid (˜0.2-0.8%), chlorphenesin (˜0.1-1.0%), caprylyl glycol (˜0.1-0.5%), aluminum stearate (˜0.1-0.6%), alumina (˜0.1-0.3%), and silica (˜0.25-3.0%), all amounts by weight, based on the weight of the composition.

In some embodiments as exemplified herein, the mineral sunscreen composition comprises tocopherol (˜0.1%), niacinamide (˜0.3%), hydroxyacetophenone (˜0.5%), citric acid (˜0.6-0.7%), chlorphenesin (˜0.2%), caprylyl glycol (˜0.3%), aluminum stearate (˜0.5-0.8%), alumina (˜0.3-0.4%), and silica (˜1%), all amounts by weight, based on the weight of the composition.

Optional additives may be selected from fragrances, pearlescent agents, silica, preservatives, proteins, protein hydrolysates, vitamins, panthenol, silicones, odor absorbers and coloring materials; anti-microbial components, including, but not limited to, phenoxyethanol, chlorphenesin, capryloyl glycol and sodium salicylate; thickeners and fillers, for example but not limited to isohexadecane (and) disteardimonium hectorite (and) propylene carbonate, and styrene/acrylates copolymer; essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang; fruit extracts, for example Pyrus malus (Apple) Fruit Extract, and Aloe barbadensis Leaf Juice Powder; citric acid, sodium chloride; neutralizing or pH-adjusting agents (e.g., triethylamine (TEA) and sodium hydroxide), and combinations thereof.

Although the optional active additives are given as examples, it will be appreciated that other optional components compatible with cosmetic applications known in the art may be used. In some particular embodiments, the mineral sunscreen composition may include antimicrobials comprising one or more of chlorphenesin and phenoxyethanol.

In some particular embodiments, the mineral sunscreen composition may include actives comprising one or more of citric acid, hydroxyacetophenone, niacinamide and tocopherol.

In various embodiments, the amount of each one or more actives and additives, when present in the mineral sunscreen composition can be present in a range from about 0.001% to about 20%, by weight, or from about 0.005% to about 0.01%, or from about 0.01% to about 0.1%, or from about 0.15% to about 5%, or from about 0.40% to about 4%, or from about 0.5% to about 2.5% by weight, or from about 1% to about 2%, or any suitable combination, sub-combination, range, or sub-range thereof by weight, based on the total weight of the mineral sunscreen composition. And in some embodiments, a combination of actives and additives present in the mineral sunscreen composition can be present in a range from about 0.001% to about 50%.

Thus, any one or a combination of actives and additives, when present, may be present, by weight, based on the total weight of the mineral sunscreen composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

### EXAMPLES

### Example 1: Raw Materials

Certain of the raw materials as used in the inventive compositions are set forth in Table 1, below.

TABLE 1
Raw Materials

| INGREDIENT | SOURCE | % Active |
|---|---|---|
| ZINC OXIDE (and) | Various | ZINC OXIDE |
| TRIETHOXYCAPRYLYLSILANE | | about 93% |
| (particle size of the ZnO | | |
| is between 20 nm to 30 nm) | | |
| TITANIUM DIOXIDE (and) | Various | TITANIUM |
| ISOHEXADECANE (and) | | DIOXIDE |
| TRIETHYLHEXANOIN (and) | | about 39% |
| ALUMINUM STEARATE (and) | | |
| ALUMINA (and) | | |
| POLYHYDROXYSTEARIC ACID | | |
| Ethylhexyl | Solastay S1(TM) | 100% |
| methoxycrylene | | |
| Diethylhexyl | Oxynex ST (TM) | 100% |
| Syringylidenemalonate | | |
| ACRYLATES/C10-30 ALKYL | Pemulen EZ-4U | 100% |
| ACRYLATE CROSSPOLYMER | (TM) | |
| XANTHAN GUM | Keltrol CG-T | 100% |
| | (TM) | |

### Example 2: Inventive Composition

Embodiments of the inventive composition are set forth in Table 2, below.

TABLE 2
Embodiments of Inventive Composition

| | Inventive | Inventive | Inventive | Inventive |
|---|---|---|---|---|
| Ingredient | Ex 1 | Ex 2 | Ex 3 | Ex 4 |
| Zinc Oxide | 15.35 | 12.38 | 10.23 | 13 |
| Titanium Dioxide | 2.65 | 4.12 | 1.77 | — |
| Emollients | 20.7 | 20.4 | 19.3 | 16.5 |
| Ethylhexyl | 2 | 2 | 2 | 2 |
| methoxycrylene | | | | |
| Diethylhexyl | 0.5 | 0.5 | — | — |
| syringylidenemalonate | | | | |
| Xanthan gum | 0.15 | 0.15 | 0.25 | 0.25 |
| Acrylates/C10-30 | 0.3 | 0.3 | 0.3 | 0.3 |
| alkyl acrylate | | | | |
| crosspolymer | | | | |
| Steareth-20 | 1 | 0.9 | 1 | 1 |
| Steareth-2 | 0.6 | 0.5 | 0.6 | 0.6 |
| Polysorbate 20 | 0.7 | 0.7 | 0.7 | 0.7 |
| Glyceryl stearate | 0.7 | 0.7 | 0.7 | 0.7 |
| Polyhydroxystearic acid | 2 | 2 | 2 | 2 |
| Glycols | 8 | 8 | 8 | 8 |
| Other ingredients | 7.2 | 7.2 | 7.0 | 7.0 |
| (aesthetic modifiers, | | | | |
| pH adjusters, | | | | |
| vitamins, | | | | |
| preservatives) | | | | |
| Water | Q.S. | Q.S. | Q.S. | Q.S. |
| Mean vivo SPF | 54.2 | 60.8 | 32.5 | 31.1 |

### Example 3: Embodiments of Inventive and Comparative Compositions Demonstrating Effect of Polymeric Thickener and Rheology Agents

TABLE 3
Aqueous Thickening Agents

| | | Additional | | Mean |
|---|---|---|---|---|
| | Anionic | Polymeric | Emulsion | vivo |
| Composition | polysaccharide | Thickener(s) | Quality | SPF |
| Inv Ex 1 | Xanthan gum | acrylates/C10-30 | Good | 54.2 |
| | (0.15%) | alkyl acrylate | | |
| | | crosspolymer (0.3%) | | |
| Comp Ex 1 | Xanthan gum | Gellan gum (0.3%) | Fail | — |
| | (0.15%) | | | |
| Comp Ex 2 | Xanthan gum | Sclerotium | Good | 34.3 |
| | (0.15%) | gum (0.3%) | | |
| Comp Ex 3 | Xanthan gum | Hydroxyethyl | Good | 35.2 |
| | (0.15%) | acrylate/sodium | | |
| | | acryloyldimethyl | | |
| | | taurate copolymer | | |
| | | (0.3%) and | | |
| | | Ammonium | | |
| | | polyacryloyldimethyl | | |
| | | taurate (0.1%) | | |
| Comp Ex 4 | Xanthan gum | Sclerotium gum | Good | 35.4 |
| | (0.15%) | (0.15%) and | | |
| | | Hydroxyethyl | | |
| | | acrylate/sodium | | |
| | | acryloyldimethyl | | |
| | | taurate copolymer | | |
| | | (0.15%) | | |

The compositions in Table 3 were prepared according to the inventive compositions in Table 2 using different aqueous phase thickening systems, whereby all compositions in Table 2 contain 18 wt % total physical UV-attenuating materials with a zinc oxide to titanium dioxide ratio of 85:15. Gellan gum is an insufficient polymeric thickener to form the emulsion structure (Comparative Ex 1). Using the polymeric thickeners in Comparative Ex 2, 3, and 4, the compositions were able to form good quality emulsions; however, were unable to achieve high SPF using the same system of physical UV-attenuating materials contained in Inventive Ex 1.

### Example 4: Zinc Oxide and Thickener Interaction and Effect on Emulsion Modulus

TABLE 4
Zinc Oxide and Thickener Relationship
in Simple Aqueous Gel Mixtures

| | Additional | Storage Modulus | Storage Modulus |
|---|---|---|---|
| | polymeric | (G′) without Zinc | after Zinc |
| Example | thickener | oxide (1%) | Oxide added |
| Inventive | Acrylates/C10-30 | 56.8 Pa | 81.3 Pa |
| polymer | alkyl acrylate | | |
| example | crosspolymer | | |
| Comparative | Sclerotium gum | 32.8 Pa | 30.8 Pa |
| polymer | | | |
| example | | | |

The examples in Table 4 were prepared as the Simple Aqueous Gel mixture in Table 5 below, whereby Storage Modulus was measured before and approximately 1 hour after adding zinc oxide (and) triethoxycaprylylsilane. Storage modulus was measured and is reported at 10% oscillation strain using a DHR-II rheometer equipped with a 40 mm sandblasted peltier parallel plate geometry at 20 degrees Celsius at 1 rad/s. The simplex aqueous gel mixture prepared using the inventive polymer example, acrylates/C10-30 alkyl acrylate crosspolymer, had a significant increase in Storage Modulus (56.8 to 81.3 Pa) after the zinc oxide material was added. Conversely, the simplex aqueous gel mixture prepared using the comparative polymer example, sclerotium gum, did not have a significant change in Storage Modulus (32.8 to 30.8 Pa) after the zinc oxide material was added. The increase in storage modulus is indicative of a structural change and specific association of acrylates/C10-30 alkyl acrylate crosspolymer and zinc oxide (and) triethoxycaprylylsilane, and is understood to be representative of the specific association of zinc oxide and an aqueous phase hydrophobically-modified anionic polymeric thickener.

TABLE 5
Simplex Aqueous Gel mixture

| Ingredient | Wt % |
|---|---|
| Zinc oxide (and) | 1% |
| triethoxycaprylylsilane | |
| Additional polymeric | 1% |
| thickener | |
| Xanthan gum | 0.5% |
| Glycerin | 11.3% |
| Propanediol | 6.8% |
| Water | Q.S. |

Together, the compositions in Table 3 and rheology data in Table 4 illustrate the important relationship of the hydrophobically-modified anionic polymeric thickener and zinc oxide.

### Example 5: Evaluation of Mineral Filters

TABLE 6
Results with Varied Mineral Filters

| | | Zinc | | | | |
|---|---|---|---|---|---|---|
| | | oxide | | | | |
| | | mean | Total | | | |
| | | primary | physical | | | White |
| | Zinc Oxide | particle | UV-attenuating | | | cast on |
| Composition | treatment | size | materials | Vivo SPF | Stability | skin |
| Inv Ex 1 | Triethoxy- | 15-70 nm | 18% | 54.2 | OK | None |
| | caprylylsilane | | | | | |
| Comp Ex 5 | Untreated | 15-70 nm | 14.5% | 49.7 | Fail after | None |
| | | | | | 2 weeks | |
| Comp Ex 6 | Silica (and) | 15-70 nm | 14.5% | 50.6 | Fail after | None |
| | hydrogen | | | | 2 weeks | |
| | dimethicone | | | | | |
| Comp Ex 7 | Triethoxy- | >100 nm | 18% | N.D. | OK | Very |
| | caprylylsilane | | | | | white |

The compositions in Table 6 illustrate the effect of the treatment and particle size of the zinc oxide material on the stability and white cast on skin. Compositions in Table 6 were prepared according to the inventive compositions in Table 2; however, the total amount of physical UV-attenuating materials and the zinc oxide materials were varied. In compositions where the total physical UV-attenuating materials were adjusted, the ratio of zinc oxide to titanium dioxide was maintained at 85:15.

White cast was measured for Inventive and Comparative compositions by applying 2 mg per cm² to an area of skin on volor forearm of about 25 cm² using light pressure over approximately 20 to 30 seconds. The relative white effect was observed after drying for at least 15 minutes on medium-to-deep skin color.

Stability was observed for inventive and comparative compositions in glass jars at temperatures ranging from 4 degrees Celsius to 50 degrees Celsius over a period of up to 4 weeks. Failure of stability indicates that significant visual separation of layers was observed in glass jars.

The important relationship of triethoxycaprylylsilane as a treatment on the zinc oxide material and the stability of the inventive composition is demonstrated in Inventive Ex 1 and Comparative Ex 7 (Table 6). However, Comparative Ex 7 contains a zinc oxide with a larger primary particle size, resulting in a significant white effect measured on skin. Untreated zinc oxide (Comparative Ex 5) or other silane treatments of zinc oxide (Comparative Ex 6) failed to result in compositions which were able to maintain acceptable stability.

While the disclosure has been described with reference to a preferred embodiment, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

### Other Definitions and Terms

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“Active material” as used herein with respect to the percent amount of an ingredient or raw material, refers to 100% activity of the ingredient in the raw material except as specifically stated. All amounts given herein are relative to the amount of active material, unless otherwise indicated.

“At least one” or “one or more” as used herein, means that there may be one, two, three or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). As used herein, the terms “comprising,” “having,” and “including” (or “comprise,” “have,” and “include”) are used in their open, non-limiting sense.

The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure.

All materials and methods described herein that embody the present disclosure can, in alternate embodiments, be more specifically defined by any of the transitional terms “comprising,” “consisting essentially of,” and “consisting of.”

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

References may be made herein are trade names for materials including, but not limited to, materials such as polymers and optional components. Materials are not intended to be limited by materials described and referenced by a certain trade name herein. Equivalent materials (e.g., those obtained from a different source under a different name or catalog (reference) number) to those referenced by trade name may be substituted and utilized in the methods described and claimed herein.

The terms “free” and “devoid” indicates that no reliably measurable excluded material is present in the mineral sunscreen composition, typically 0% by weight, based on the total weight of the mineral sunscreen composition.

As used herein, the terms “substantially free” or “essentially free” as used herein mean the specific material may be present in small amounts that do not materially affect the basic and novel characteristics of the embodiments of the mineral sunscreen composition according to the disclosure or the material may be absent. For instance, there may be less than 2% by weight of a specific material added to a composition, based on the total weight of the mineral sunscreen compositions (provided that an amount of less than 2% by weight does not materially affect the basic and novel characteristics of embodiments of the mineral sunscreen composition according to the disclosure. Similarly, the mineral sunscreen compositions may include less than 2%, less than 1.5%, less than 1%, less than 0.5%, less than 0.1%, less than 0.05%, or less than 0.01%, or none (0%) of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the mineral sunscreen composition but may still be present in a raw material that is included in the mineral sunscreen composition.

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in the mineral sunscreen composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A mineral sunscreen composition, comprising:
a broad spectrum, low viscosity, oil-in-water emulsion comprising aqueous and oil phases, the mineral sunscreen composition comprising:
a) a UV absorbing system comprising at least one physical UV attenuating material, the at least one physical UV attenuating material comprising zinc oxide primary particles having a mean size in a range from about 15 nm to about 35 nm, the zinc oxide particles coated with triethoxycaprylylsilane;
b) an emulsification system comprising:
i. at least one high HLB emulsifier having an HLB greater than 13;
ii. at least one low HLB emulsifier having an HLB of less than 6; and
c) an aqueous phase hydrophobically-modified anionic polymeric thickener comprising carboxylic acid residues;
wherein the combined high HLB and low HLB emulsifiers to hydrophobically-modified anionic polymeric thickener are present in a ratio in a range from about 1.0:0.05 to about 1.0:0.15, and wherein the mineral sunscreen composition demonstrates an SPF that is at least 40, a UVA-I/UV ratio that is at least 0.7, and a critical wavelength that is greater than 370 nm.

2. The mineral sunscreen composition according to claim 1, characterized by one or more of the properties selected from:
a) a critical wavelength as determined by FDA's critical wavelength procedures of at least 370 nm;
b) an SPF value of at least 15; or
c) combinations thereof.

3. The mineral sunscreen composition according to claim 1, wherein the composition has an SPF with a value of at least 30.

4. The mineral sunscreen composition according to claim 1, wherein the composition has an SPF with a value of at least 50.

5. The mineral sunscreen composition according to claim 1, wherein the mineral sunscreen composition excludes organic UV filters.

6. The mineral sunscreen composition according to claim 1, wherein the zinc oxide particles are selected from the group consisting of zinc oxide (and) triethoxycaprylylsilane, having a particle size of about 20 nm, zinc oxide (and) silica (and) hydrogen dimethicone, having a particle size of about 25 nm, zinc oxide (and) caprylic/capric triglyceride (and) polyhydroxystearic acid (and) isostearic acid, having a particle size of about 60 nm, zinc oxide (and) C12-15 alkyl benzoate (and) triethoxycaprylylsilane (and) polyhydroxystearic acid, having a particle size of about 30 nm, and combinations thereof.

7. The mineral sunscreen composition according to claim 1, wherein the at least one physical UV attenuating material is present in the mineral sunscreen composition in the range from about 10% to about 25%, all amounts based on the total weight of the mineral sunscreen composition.

8. The mineral sunscreen composition according to claim 1, wherein the aqueous phase hydrophobically-modified polymeric thickener further functions as a polymeric emulsifier.

9. The mineral sunscreen composition according to claim 1, wherein the aqueous phase hydrophobically-modified polymeric thickener comprises acrylates/C10-30 alkyl acrylate crosspolymer.

10. The mineral sunscreen composition according to claim 1, wherein the emulsification system comprises glyceryl stearate, steareth-2, steareth-20, and polysorbate 20.

11. The mineral sunscreen composition according to claim 1, comprising a rheology modifier selected from the group consisting of xanthan gum, cellulose gum, biosaccharide gum, carrageenan, gellan gum, or associative thickeners such as like Sepinov EMT10™ or Sepigel™, Aristoflex™, and combinations thereof.

12. The mineral sunscreen composition according to claim 1, comprising at least one antioxidant comprising one or more of diethylhexyl syringylidenemalonate and ethylhexyl methoxycrylene.

13. The mineral sunscreen composition according to claim 1, comprising an oil phase solvent system comprising one or a combination of oil/fatty compounds and silicones selected from caprylic/capric triglyceride, cetearyl isononanoate, cetearyl alcohol, dicaprylyl carbonate, isohexadecane, C15-19 alkane, dimethicone, and combinations thereof.

14. The mineral sunscreen composition according to claim 1, comprising one or more additives selected from the group consisting of skin conditioners, hydrating agents, skin actives, preservatives, antimicrobials, powders, fillers, and combinations thereof.

15. The mineral sunscreen composition according to claim 1, comprising one or more additives selected from the group consisting of triethylhexanoin, glycerin, propanediol, tocopherol, niacinamide, hydroxyacetophenone, citric acid, chlorphenesin, caprylyl glycol, aluminum stearate, alumina, silica, cellulose, and combinations thereof.

16. The mineral sunscreen composition according to claim 1, wherein the components of the aqueous phase and components of the oil phase, excluding the physical UV attenuating materials and any powders, respectively, are present in the mineral sunscreen composition at a weight ratio of aqueous components to oil components in a range from about 1:1 to about 4:1

17. A metastable structured emulsion comprising an aqueous phase hydrophobically-modified polymeric thickener comprising acrylates/C10-30 alkyl acrylate crosspolymer and a physical UV attenuating material comprising one or more of zinc oxide and/or titanium dioxide, the one or more of zinc oxide and/or titanium dioxide comprising a coating selected from hydrated silica, triethoxysilylethyl polydimethylsiloxyethyl, hexyl dimethicone, hydrogen dimethicone, triethoxycaprylysilane, or a combination thereof, the metastable structured emulsion formed through the interaction of carboxylic acid residues of the aqueous phase hydrophobically-modified polymeric thickener with the coated zinc oxide and/or titanium dioxide, which emulsion provides a high SPF that does not whiten on application by rubbing on a substrate such as skin.

18. A mineral sunscreen composition in the form of a broad spectrum, low viscosity, oil-in-water emulsion comprising aqueous and oil phases, the mineral sunscreen composition comprising:
a) a UV absorbing system comprising at least one physical UV attenuating material, the at least one physical UV attenuating material comprising zinc oxide particles having a primary particle size in a range from about 15 nm to about 35 nm, the zinc oxide particles coated with triethoxycaprylylsilane, and titanium dioxide;
b) an emulsification system comprising at least one high HLB emulsifier having an HLB of greater than 13 comprising steareth-20 and polysorbate 20 and at least one low HLB emulsifier having an HLB of less than 6, comprising glyceryl stearate and steareth-2;
c) at least one aqueous phase hydrophobically-modified anionic polymeric thickener comprising acrylates/C10-30 alkyl acrylate crosspolymer;
d) at least one polymeric dispersant comprising polyhydroxystearic acid;
e) at least one rheology modifier comprising xanthan gum;
f) at least one antioxidant comprising diethylhexyl syringylidenemalonate and ethylhexyl methoxycrylene;
g) an oil phase solvent system comprising caprylic/capric triglyceride, cetearyl isononanoate, cetearyl alcohol, dicaprylyl carbonate, isohexadecane, C15-19 alkane, and dimethicone; and
h) one or more additives selected from the group consisting of triethylhexanoin, glycerin, propanediol, tocopherol, niacinamide, hydroxyacetophenone, citric acid, chlorphenesin, caprylyl glycol, aluminum stearate, alumina, silica, cellulose, and combinations thereof,
wherein the combined high HLB and low HLB emulsifiers to hydrophobically-modified anionic polymeric thickener are present in a ratio in a range from about 1.0:0.05 to about 1.0:0.15, and wherein the mineral sunscreen composition demonstrates an SPF that is at least 40, a UVA-I/UV ratio that is at least 0.7, and a critical wavelength that is greater than 370 nm.
